(19) United States
(12) Patent Application Publication
(10) Pub. No.: US 2026/0108601 A1
(43) Pub. Date: Apr. 23, 2026

(54) METHOD OF DIAGNOSIS AND TREATMENT OF RHEUMATOID ARTHRITIS

(71) Applicants: Sanofi Biotechnology, Gentilly (FR); Regeneron Pharmaceuticals, Inc., Tarrytown, NY (US)

(72) Inventors: Anita Boyapati, Chappaqua, NY (US); Jérôme Msihid, Antony (FR); Moshe E. Zilberstein, North Caldwell, NJ (US)

(21) Appl. No.: 19/336,021

(22) Filed: Sep. 22, 2025

Related U.S. Application Data
(63) Continuation of application No. 16/858,301, filed on Apr. 24, 2020, now Pat. No. 12,440,561.
(60) Provisional application No. 62/837,793, filed on Apr. 24, 2019.

(30) Foreign Application Priority Data
Feb. 27, 2020 (EP) 20305193.3

(51) Int. Cl. A61K 39/395; A61B 5/00; A61K 9/08; A61K 31/519; A61P 19/02; C07K 16/28

(52) U.S. Cl. CPC A61K 39/3955; A61B 5/4848; A61K 9/08; A61K 31/519; A61P 19/02; C07K 16/2866; C07K 2317/51; C07K 2317/515

(57) ABSTRACT

The present disclosure relates to the use of an anti-IL6 receptor antibody for treating rheumatoid arthritis in subjects with serum concentrations of certain biomarkers.

### RELATED APPLICATIONS

This application is a continuation of U.S. patent application Ser. No. 16/858,301, filed Apr. 24, 2020, which claims the benefit of U.S. Provisional Patent Application No. 62/837,793, filed Apr. 24, 2019; and European Patent Application No. 20305193.3, filed on Feb. 27, 2020; each of which is incorporated herein by reference in its entirety.

### SEQUENCE LISTING

The instant application contains a Sequence Listing which has been submitted electronically in XML format and is hereby incorporated by reference in its entirety. Said XML file, created on Sep. 18, 2025, is named 769956_SA9-264CON_ST26.xml and is 47,081 bytes in size.

### FIELD

The present disclosure relates to the field of treating rheumatoid arthritis in a subject with serum concentrations of certain biomarkers, by administering to the subject an anti-IL6 receptor antibody.

### BACKGROUND

Patients with rheumatoid arthritis (RA) develop bone and cartilage damage in synovial joints as a result of chronic inflammation, which is mediated by pro-inflammatory cytokines such as interleukin-6 (IL-6) and tumour necrosis factor alpha (TNF-α). In RA, elevated circulating cytokine concentrations trigger bone and cartilage destruction through activation of signaling cascades that lead to the stimulation of osteoclasts via bone-resorptive factors (e.g. receptor activator of nuclear factor-κB ligand [RANKL]) and joint-destructive proteins (e.g. matrix metalloproteinases). Underlying joint damage leads to long-term impairments in physical function.

IL-6 is a pleiotropic cytokine that plays a role in inflammatory, metabolic, neural and regenerative processes. IL-6 operates through two distinct mechanisms-classic (cis) and trans-signaling-which expands the range of its actions and contributes towards the systemic manifestations and co-morbidities commonly associated with RA, including the acute-phase response, osteoporosis, fatigue, depression, anemia and cardiovascular (CV) disease. Patients with RA have an increased risk of CV events, including myocardial infarction and stroke, relative to healthy individuals. It is understood that pro-inflammatory cytokines promote endothelial dysfunction and structural vessel abnormalities, and induce other CV risk factors, including changes in lipid levels, insulin resistance, and oxidative stress. In addition, significantly elevated levels of lipoprotein (a) (Lp[a]), a biomarker of CV risk which is involved in both inflammation and thrombosis, have been observed in patients with RA compared with healthy controls.

Sarilumab is a human monoclonal antibody that binds membrane-bound and soluble IL-6 receptor-α to inhibit IL-6 signaling. It is approved for the treatment of adults with moderate-to-severe active RA as monotherapy and in combination with conventional synthetic disease-modifying anti-rheumatic drugs (csDMARDs). There is a need to define patient populations that show better efficacy with sarilumab when treating RA so that they are administered the drug sooner.

### SUMMARY

This disclosure provides, inter alia, methods for identifying subjects with a propensity to respond to treatment with an anti-interleukin 6 receptor (IL-6R) antibody, such as an antibody comprising a heavy chain variable region comprising the sequence SEQ ID NO: 1 and a light chain variable region comprising the sequence SEQ ID NO:2 (e.g., sarilumab). In some embodiments, the methods identify the subject as having a higher propensity to respond to an IL-6R antibody (such as sarilumab) than a TNF-α inhibitor (such as adalimumab). The present disclosure further provides methods of treating RA in subjects who have levels of certain biomarkers that identify the subject as having a propensity to respond to administration of an IL-6R antibody

In an aspect, provided herein is a method of treating rheumatoid arthritis in a subject with a serum concentration of C-reactive protein (CRP) of greater than 27.9 mg/L comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, the subject has a serum concentration of CRP equal to or greater than 37.6 mg/L. In some embodiments, the subject has a serum concentration of CRP equal to or greater than 65.1 mg/L.

In some embodiments, a method of treating rheumatoid arthritis in a subject with a serum concentration of C-reactive protein (CRP) greater than 27.9 mg/L comprises selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously.

In some embodiments, the serum concentration of CRP is less than 65.1 mg/L in the subject. In some embodiments, the subject achieves ACR20 after 24 weeks of treatment with the antibody.

In some embodiments, the subject achieves a DAS28-CRP score of less than 3.2 after 24 weeks of treatment with the antibody. In some embodiments, the subject achieves an improvement in patient global VAS, HAQ-DI, Pain VAS, SF-36-PCS score or morning stiffness VAS after 24 weeks of treatment with the antibody.

This disclosure further provides a method of treating rheumatoid arthritis in a subject with a serum concentration of C-reactive protein (CRP) greater than 6.9 mg/L and less than 13.1 mg/L comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of C-reactive protein (CRP) greater than 6.9 mg/L and less than 13.1 mg/L comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO:2 and the antibody is administered subcutaneously.

In some embodiments, the subject achieves an improvement in patient global VAS, Pain VAS, SF-36-PCS score, SF-36-RE domain, morning stiffness VAS or RAID score after 24 weeks of treatment with the antibody.

This disclosure further provides a method of treating rheumatoid arthritis in a subject with a serum concentration of serum amyloid A (SAA) greater than 105.2 ng/ml comprising selecting the subject and administering to the subject an anti-IL-6R antibody.

In some embodiments, the subject has a serum concentration of SAA less than 256.0 ng/mL. In some embodiments, the antibody comprises a heavy chain variable region comprising the sequence SEQ ID NO:1 and a light chain variable region comprising the sequence SEQ ID NO: 2. In some embodiments, the antibody is sarilumab. In some embodiments, the antibody is administered subcutaneously.

In some embodiments, the subject has a serum concentration of SAA equal to or greater than 174.9 ng/mL. In some embodiments, the subject has a serum concentration of SAA equal to or greater than 256.0 ng/mL.

In some embodiments, a method of treating rheumatoid arthritis in a subject with a serum concentration of serum amyloid A (SAA) greater than 105.2 ng/ml, comprising selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously. In some embodiments, the subject has a serum concentration of SAA less than 256.0 ng/mL.

In some embodiments, the subject achieves ACR20, ACR50 or ACR70 after 24 weeks of treatment with the antibody. In some embodiments, the subject achieves a DAS28-CRP score of less than 3.2 after 24 weeks of treatment with the antibody. In some embodiments, wherein the subject achieves an improvement in patient global VAS, HAQ-DI, Pain VAS, SF-36-PCS score, SF-36-PF domain, morning stiffness VAS or RAID score after 24 weeks of treatment with the antibody.

This disclosure further provides a method of treating rheumatoid arthritis in a subject with a serum concentration of SAA greater than 11.83 ng/ml and less than 30.08 ng/ml comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of serum amyloid A (SAA) greater than 11.83 ng/ml and less than 30.08 ng/ml, comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO:2 and the antibody is administered subcutaneously.

In some embodiments, the subject achieves an improvement in HAQ-DI after 24 weeks of treatment with the antibody.

This disclosure further provides a method of treating rheumatoid arthritis in a subject with a serum concentration of matrix metalloproteinase-3 (MMP-3) greater than 77.0 ng/ml comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, the subject has a serum concentration of MMP-3 equal to or greater than 99.9 ng/mL. In some embodiments, the subject has a serum concentration of MMP-3 equal to or greater than 154.3 ng/mL.

In some embodiments, a method of treating rheumatoid arthritis in a subject with a serum concentration of matrix metalloproteinase-3 (MMP-3) greater than 77.0 ng/ml, comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO: 2 and the antibody is administered subcutaneously.

In some embodiments, the serum concentration of MMP-3 is less than 154.3 ng/ml in the subject. In some embodiments, the subject achieves ACR20 after 24 weeks of treatment with the antibody. In some embodiments, the subject achieves a DAS28-CRP score of less than 3.2 after 24 weeks of treatment with the antibody. In some embodiments, the subject achieves an improvement in patient global VAS, HAQ-DI, Pain VAS, SF-36-PCS score, SF-36-PF domain, morning stiffness VAS or RAID score after 24 weeks of treatment with the antibody.

This disclosure further provides a method of treating rheumatoid arthritis in a subject with a serum concentration of MMP-3 greater than 35.5 ng/ml and less than 54.1 ng/ml comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of matrix metalloproteinase-3 (MMP-3) greater than 35.5 ng/mL and less than 54.1 ng/mL, comprises selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously.

In some embodiments, the subject achieves an improvement in pain VAS after 24 weeks of treatment with the antibody.

This disclosure further provides a method of treating rheumatoid arthritis in a subject with a serum concentration of chemokine (C—X—C motif) ligand 13 (CXCL13) greater than 180.8 pg/mL comprising selecting the subject and administering to the subject an anti-IL-6R antibody.

In some embodiments, the antibody comprises a heavy chain variable region comprising the sequence SEQ ID NO:1 and a light chain variable region comprising the sequence SEQ ID NO: 2. In some embodiments, the antibody is sarilumab. In some embodiments, the antibody is administered subcutaneously.

In some embodiments, the subject has a serum concentration of CXCL13 equal to or greater than 236.8 pg/mL. In some embodiments, the subject has a serum concentration of CXCL13 equal to or greater than 323.9 pg/mL.

In some embodiments, a method of treating rheumatoid arthritis in a subject with a serum concentration of chemokine (C—X—C motif) ligand 13 (CXCL13) greater than 180.8 pg/mL, comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO:2 and the antibody is administered subcutaneously.

In some embodiments, the serum concentration of CXCL3 is less than 323.9 pg/mL in the subject. In some embodiments, the subject achieves ACR20 after 24 weeks of treatment with the antibody. In some embodiments, the subject achieves an improvement in HAQ-DI, SF-36-PCS score or SF-36-PF domain after 24 weeks of treatment with the antibody.

This disclosure further provides a method of treating rheumatoid arthritis in a subject with a serum concentration of CXCL13 greater than 98.2 pg/mL and less than 130.6 pg/ml comprising selecting the subject and administering to the subject an anti-IL-6R antibody.

In some embodiments, the antibody comprises a heavy chain variable region comprising the sequence SEQ ID NO:1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of chemokine (C—X—C motif) ligand 13 (CXCL13) greater than 98.2 pg/mL and less than 130.6 pg/ml comprises selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously.

In some embodiments, the subject achieves an improvement in HAQ-DI after 24 weeks of treatment with the antibody.

This disclosure further provides a method of treating rheumatoid arthritis in a subject with a serum concentration of hepcidin greater than 43.9 ng/ml comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, the subject has a serum concentration of hepcidin equal to or greater than 62.4 ng/mL. In some embodiments, the subject has a serum concentration of hepcidin equal to or greater than 77 ng/mL.

In some embodiments, a method of treating rheumatoid arthritis in a subject with a serum concentration of hepcidin greater than 62.4 ng/mL, comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO:2 and the antibody is administered subcutaneously.

In some embodiments, the serum concentration of hepcidin is less than 77 ng/ml in the subject. In some embodiments, the subject achieves ACR20 after 24 weeks of treatment with the antibody.

This disclosure further provides a method of treating rheumatoid arthritis in a subject with a serum concentration of hepcidin greater than 17 ng/mL and less than 28.9 ng/ml comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of hepcidin greater than 17 ng/ml and less than 28.9 ng/ml comprises selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously.

In some embodiments, the subject achieves an improvement in HAQ-DI after 24 weeks of treatment with the antibody.

This disclosure further provides a method of treating rheumatoid arthritis in a subject with a serum concentration of CXCL13 less than 72.0 pg/mL and a serum concentration of soluble intracellular adhesion molecule-1 (sICAM-1) of less than 212.1 ng/mL comprising selecting the subject and administering to the subject an anti-IL-6R antibody.

In some embodiments, the antibody comprises a heavy chain variable region comprising the sequence SEQ ID NO: 1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of chemokine CXCL13 less than 72.0 pg/mL and a serum concentration of soluble intracellular adhesion molecule-1 (SICAM-1) of less than 212.1 ng/ml comprises selecting the subject and administering to the subject about 150 mg to about 200 mg of an antibody once every two weeks, wherein the antibody comprises a heavy chain variable region comprising the sequence SEQ ID NO:1 and a light chain variable region comprising the sequence SEQ ID NO:2 and the antibody is administered subcutaneously.

In some embodiments, the serum concentration of CXCL3 is greater than 52.4 pg/mL in the subject. In some embodiments, serum concentration of sICAM1 is greater than 179.7 ng/mL in the subject. In some embodiments, the subject achieves ACR50 after 24 weeks of treatment with the antibody.

This disclosure further provides a method of treating rheumatoid arthritis in a subject with a serum concentration of CXCL13 greater than 180.8 pg/mL and a serum concentration of SICAM-1 of greater than 313.7 ng/mL comprising selecting the subject and administering to the subject an anti-IL-6R antibody.

In some embodiments, the antibody comprises a heavy chain variable region comprising the sequence SEQ ID NO:1 and a light chain variable region comprising the sequence SEQ ID NO: 2. In some embodiments, the antibody is sarilumab. In some embodiments, the antibody is administered subcutaneously.

In some embodiments, a method of treating rheumatoid arthritis in a subject with a serum concentration of chemokine CXCL13 greater than 180.8 pg/mL and a serum concentration of SICAM-1 of greater than 313.7 ng/ml comprises selecting the subject and administering to the subject about 150 mg to about 200 mg of an antibody once every two weeks, wherein the antibody comprises a heavy chain variable region comprising the sequence SEQ ID NO: 1 and a light chain variable region comprising the sequence SEQ ID NO:2 and the antibody is administered subcutaneously.

In some embodiments, the serum concentration of CXCL3 is less than 323.9 pg/mL in the subject. In some embodiments, the serum concentration of sICAM-1 is less than 380.0 ng/ml in the subject. In some embodiments, the subject achieves ACR50 after 24 weeks of treatment with the antibody. In some embodiments of any of the methods provided above, the subject has moderate to severe rheumatoid arthritis. In some embodiments, the antibody is administered with a prefilled syringe or with an auto-injector.

In some embodiments, the antibody is administered as an aqueous buffered solution at about pH 6.0 containing about 21 mM histidine, about 45 mM arginine, about 0.2% (w/v) polysorbate 20, and about 5% (w/v) sucrose. In some embodiments, the solution comprises at least about 130 mg/mL of the antibody. In some embodiments, the solution comprises about 131.6 mg/mL of the antibody. In some embodiments, the solution comprises about 175 mg/ml of the antibody. In some embodiments, the antibody is sarilumab. In some embodiments, the subject is intolerant of one or more DMARDs. In some embodiments, the DMARD is methotrexate. In some embodiments, the method further comprises administering to the subject an effective amount of methotrexate. In some embodiments, methotrexate is administered between 6 to 25 mg per week. In some embodiments, the subject has moderate to severe rheumatoid arthritis and has had an inadequate response to one or more DMARDs. In some embodiments, the DMARD is methotrexate. In some embodiments, the antibody is administered subcutaneously at 150 mg once every two weeks to the subject. In some embodiments, the antibody is administered subcutaneously at 200 mg once every two weeks to the subject.

This disclosure further provides a method of determining if a subject with rheumatoid arthritis has an increased propensity for effective treatment of rheumatoid arthritis with an antibody comprising measuring a concentration of CRP in a serum sample from the subject wherein if the serum sample has a concentration of CRP greater than 27.9 mg/L, 37.6 mg/L, or 65.1 mg/L, the subject has an increased propensity for effective treatment of RA with the antibody, and wherein the antibody comprises a heavy chain variable region comprising the sequence SEQ ID NO:1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the serum sample from the subject has a concentration of CRP less than 65.1 mg/L in the subject. In some embodiments, the subject has an increased propensity of achieving ACR20 after 24 weeks of treatment with the antibody. In some embodiments, the subject has an increased propensity of achieving a DAS28-CRP score of less than 3.2 after 24 weeks of treatment with the antibody. In some embodiments, the subject has an increased propensity of achieving an improvement in patient global VAS, Pain VAS, SF-36-PCS score, SF-36-RE domain, morning stiffness VAS or RAID score after 24 weeks of treatment with the antibody.

This disclosure further provides a method of determining if a subject with rheumatoid arthritis has an increased propensity for effective treatment of rheumatoid arthritis with an antibody comprising measuring a concentration of CRP in a serum sample from the subject wherein if the serum sample has a concentration of CRP greater than 6.9 mg/L and less than 13.1 mg/L, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the subject has an increased propensity of achieving an improvement in patient global VAS, Pain VAS, SF-36-PCS score, SF-36-RE domain, morning stiffness VAS or RAID score after 24 weeks of treatment with the antibody.

This disclosure further provides a method of determining if a subject with rheumatoid arthritis has an increased propensity for effective treatment of rheumatoid arthritis with an antibody comprising measuring a concentration of SAA in a serum sample from the subject wherein if the serum sample has a concentration of SAA greater than 105.2 ng/ml, 174.9 ng/ml, or 256 ng/ml and less than 256.0 ng/ml, the subject has an increased propensity for effective treatment of RA with the antibody, and wherein the antibody comprises a heavy chain variable region comprising the sequence SEQ ID NO:1 and a light chain variable region comprising the sequence SEQ ID NO: 2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the subject has an increased propensity of achieving ACR20, ACR50 or ACR70 after 24 weeks of treatment with the antibody. In some embodiments, wherein the subject has an increased propensity of achieving a DAS28-CRP score of less than 3.2 after 24 weeks of treatment with the antibody. In some embodiments, wherein the subject has an increased propensity of achieving an improvement in patient global VAS, HAQ-DI, Pain VAS, SF-36-PCS score, SF-36-PF domain, morning stiffness VAS or RAID score after 24 weeks of treatment with the antibody.

This disclosure further provides a method of determining if a subject with rheumatoid arthritis has an increased propensity for effective treatment of rheumatoid arthritis with an antibody comprising measuring a concentration of SAA in a serum sample from the subject wherein if the serum sample has a concentration of SAA greater than 11.83 ng/ml and less than 30.08 ng/ml, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the subject has an increased propensity of achieving an improvement in HAQ-DI after 24 weeks of treatment with the antibody.

This disclosure further provides a method of determining if a subject with rheumatoid arthritis has an increased propensity for effective treatment of rheumatoid arthritis with an antibody comprising measuring a concentration of MMP-3 in a serum sample from the subject wherein if the serum sample has a concentration of MMP-3 greater than 77.0 ng/ml, 99.9 ng/mL, or 154.3 ng/mL, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the serum sample from the subject has a concentration of MMP-3 is less than 154.3 ng/ml in the subject. In some embodiments, the subject has an increased propensity of achieving ACR20 after 24 weeks of treatment with the antibody. In some embodiments, the subject has an increased propensity of achieving a DAS28-CRP score of less than 3.2 after 24 weeks of treatment with the antibody. In some embodiments, the subject has an increased propensity of achieving an improvement in patient global VAS, HAQ-DI, Pain VAS, SF-36-PCS score, SF-36-PF domain, morning stiffness VAS or RAID score after 24 weeks of treatment with the antibody.

This disclosure further provides a method of determining if a subject with rheumatoid arthritis has an increased propensity for effective treatment of rheumatoid arthritis with an antibody comprising measuring a concentration of MMP-3 in a serum sample from the subject wherein if the serum sample has a concentration of MMP-3 greater than 35.5 ng/ml and less than 54.1 ng/ml, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the subject has an increased propensity of achieving an improvement in pain VAS after 24 weeks of treatment with the antibody.

This disclosure further provides a method of determining if a subject with rheumatoid arthritis has an increased propensity for effective treatment of rheumatoid arthritis with an antibody comprising measuring a concentration of CXCL13 in a serum sample from the subject wherein if the serum sample has a concentration of CXCL13 greater than 98.2 pg/mL, 116.4 pg/mL, 130.6 pg/mL, 180.8 pg/mL, 236.8 pg/mL, or 323.9 pg/mL, the subject has an increased propensity for effective treatment of RA with the antibody, and wherein the antibody comprises a heavy chain variable region comprising the sequence SEQ ID NO:1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the serum sample from the subject has a concentration of CXCL3 is less than 323.9 pg/mL in the subject. In some embodiments, the subject has an increased propensity of achieving ACR20 after 24 weeks of treatment with the antibody. In some embodiments, the subject has an increased propensity of achieving an improvement in HAQ-DI, SF-36-PCS score or SF-36-PF domain after 24 weeks of treatment with the antibody.

This disclosure further provides a method of determining if a subject with rheumatoid arthritis has an increased propensity for effective treatment of rheumatoid arthritis with an antibody comprising measuring a concentration of hepcidin in a serum sample from the subject wherein if the serum sample has a concentration of hepcidin greater than 17 ng/ml, 23 ng/ml, 28.9 ng/ml, 43.9 ng/ml, 62.4 ng/ml, or 77 ng/ml, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the subject has an increased propensity of achieving ACR20 after 24 weeks of treatment with the antibody. This disclosure further provides a method of determining if a subject with rheumatoid arthritis has an increased propensity for effective treatment of rheumatoid arthritis with an antibody comprising measuring a concentration of CXCL13 in a serum sample from the subject wherein if the serum sample has a concentration of CXCL13 less than 72.0 pg/mL and a concentration of sICAM-1 of less than 212.1 ng/ml, the subject has an increased propensity for effective treatment of RA with the antibody, and wherein the antibody comprises a heavy chain variable region comprising the sequence SEQ ID NO: 1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the serum sample from the subject has a concentration of CXCL3 is greater than 52.4 pg/mL in the subject. In some embodiments, the serum sample from the subject has a concentration of sICAM1 is greater than 179.7 ng/mL in the subject. In some embodiments, the subject has an increased propensity of achieving ACR50 after 24 weeks of treatment with the antibody.

This disclosure further provides a method of determining if a subject with rheumatoid arthritis has an increased propensity for effective treatment of rheumatoid arthritis with an antibody comprising measuring a concentration of CXCL13 in a serum sample from the subject wherein if the serum sample has a concentration of CXCL13 greater than 180.8 pg/mL and a concentration of sICAM-1 of greater than 313.7 ng/mL, the subject has an increased propensity for effective treatment of RA with the antibody, and wherein the antibody comprises a heavy chain variable region comprising the sequence SEQ ID NO:1 and a light chain variable region comprising the sequence SEQ ID NO:2. In some embodiments, the subject has an increased propensity for effective treatment of rheumatoid arthritis with an anti-IL-6R antibody (such as sarilumab) compared to a TNF-α inhibitor (such as adalimumab).

In some embodiments, the serum sample from the subject has a concentration of CXCL3 is less than 323.9 pg/mL in the subject. In some embodiments, the serum sample from the subject has a concentration of sICAM-1 is less than 380.0 ng/ml in the subject. In some embodiments, the subject has an increased propensity of achieving ACR50 after 24 weeks of treatment with the antibody. In some embodiments of the methods of determining if a subject with rheumatoid arthritis has an increased propensity for effective treatment described above, the subject has moderate to severe rheumatoid arthritis. In some embodiments, the antibody is sarilumab. In some embodiments, the subject intolerant of one or more DMARDs. In some embodiments, the DMARD is methotrexate. In some embodiments, the subject has moderate to severe rheumatoid arthritis and has had an inadequate response to one or more DMARDs. In some embodiments, the DMARD is methotrexate.

In some embodiments, a serum sample is from the peripheral blood of a subject.

In some embodiments, the subject has at least 5, 6, 7, 8, 9, or 10 tender joints. In some embodiments, the subject has at least 10, 15, 20, 25, or 30 tender joints. In some embodiments, the subject has 5-10, 10-15, 15-20, or 10-20 tender joints. In some embodiments, the subject has at least 5, 6, 7, 8, 9, or 10 swollen joints. In some embodiments, the subject has at least 10, 15, 20, 25, or 30 swollen joints. In some embodiments, the subject has 5-10, 10-15, 15-20, or 10-20 swollen joints. In some embodiments, the subject has joint erosion in at least 1, 2, 3, 4, 5, 6, 7, 8, 9, or 10 joints. In some embodiments, the subject has joint erosion in 1-5, 5-10, 1-10, 10-15, or 1-15 joints. In some embodiments, the subject has a Disease Activity Score using 28 Joints (DAS28) of 5, 6, 7, 8, 9, or 10. In some embodiments, the subject has a DAS28 score of at least 5. In some embodiments, the subject has a DAS28 score of at least 6. In some embodiments, the subject has a DAS28 score of at least 7. In some embodiments, the subject has a DAS28 score of at least 8. In some embodiments, the subject has a DAS28 score of at least 9. In some embodiments, the subject has a DAS28 score of at least 10. In some embodiments, the subject has a DAS28 score of 5-10.

### BRIEF DESCRIPTION OF THE DRAWINGS

FIGS. 1A-1B depict the correlation between baseline biomarkers and haematology parameters. CRP: C-reactive protein; CXCL13: chemokine (C—X—C motif) ligand 13; Lp(a): lipoprotein (a); MMP-3: matrix metalloproteinase-3; OPG: osteoprotegerin; PINP: procollagen type 1 N-terminal propeptide; RANKL: receptor activator of nuclear factor-κB ligand; SAA: serum amyloid A; SICAM-1: soluble intercellular adhesion molecule 1; TIBC: total iron-binding capacity.

FIGS. 2A-2C depict the median percentage changes from baseline in biomarkers through week 24. Median percentage changes from baseline in biomarkers of (FIGS. 2A and 2B) the acute-phase response (CRP and SAA) and (FIG. 2C) bone remodeling (PINP). *Adjusted P<0.05; **adjusted P<0.01 vs adalimumab; ***adjusted P<0.0001 vs adalimumab (Benjamini-Hochberg procedure). CRP: C-reactive protein; PINP: procollagen type 1 N-terminal propeptide; q2w: every 2 weeks; SAA: serum amyloid A.

FIGS. 3A-3D depict the median percentage changes from baseline in biomarkers through week 24. Median percentage changes from baseline in biomarkers of (FIGS. 3A-3C) bone remodeling (OC, total RANKL and OPG), (FIG. 3D) synovial inflammation (MMP-3). *Adjusted P<0.05; **adjusted P<0.01 vs adalimumab; ***adjusted P<0.0001 vs adalimumab (Benjamini-Hochberg procedure). MMP-3: matrix metalloproteinase-3; OC: osteocalcin; OPG: osteoprotegerin; q2w: every 2 weeks; RANKL: receptor activator of nuclear factor-κB ligand.

FIG. 4 depicts the median percentage changes from baseline CXCL13 and sICAM-1 through week 24. ***Adjusted P<0.0001 vs adalimumab (Benjamini-Hochberg procedure). CXCL13: chemokine (C X-C motif) ligand 13; Q: quartile; q2w: every 2 weeks; SICAM-1: soluble intercellular adhesion molecule-1.

FIGS. 5A-5D depict the median percentage changes from baseline in biomarkers of anemia of chronic disease 2 weeks post-treatment. Median percentage changes from baseline in (FIG. 5A) hepcidin, (FIG. 5B) iron, (FIG. 5C) ferritin, and (FIG. 5D) TIBC at week 2 post-treatment. ***Adjusted P<0.0001 vs adalimumab (Benjamini-Hochberg procedure). **Adjusted P<0.01 vs adalimumab (Benjamini-Hochberg procedure). Q: quartile; q2w: every 2 weeks; TIBC: total iron-binding capacity.

FIGS. 6A-6B depict (FIG. 6A) the median percentage changes from baseline in atherothrombosis (Lp[a]) through week 24 and (FIG. 6B) the proportions of patients whose biomarker values returned to normal reference ranges at week 24. In FIG. 6A, *Adjusted P<0.05; **adjusted P<0.01 vs adalimumab; ***adjusted P<0.0001 vs adalimumab (Benjamini-Hochberg procedure). In FIG. 6B, proportions of patients with biomarker serum concentrations exceeding the reference range at baseline that normalized to within reference range at week 24. ***Unadjusted P<0.0001 vs adalimumab (χ2 test). CRP: C-reactive protein; Lp(a): lipoprotein (a); OC: osteocalcin; OPG: osteoprotegerin; PINP: procollagen type 1 N-terminal propeptide; q2w: every 2 weeks; RANKL: receptor activator of nuclear factor-κB ligand; SAA: serum amyloid A.

FIGS. 7A-7B depict (FIG. 7A) the odds ratios for achieving efficacy endpoints at week 24 by baseline biomarker tertiles and (FIG. 7B) the changes from baseline in patient reported outcomes (PROs) at week 24 by baseline biomarker tertiles. In FIG. 7A, odds ratios (sarilumab vs adalimumab) for achieving ACR20, ACR50 and DAS28-CRP <3.2 responses at week 24 by baseline biomarker tertiles. *Nominal biomarker-by-treatment interaction vs low tertile. Low, medium and high subgroups are based on biomarker tertile values in overall treatment groups (see Table 1 for tertile ranges). ACR20/50: American College of Rheumatology 20/50% improvement criteria; CI: confidence interval; DAS28-CRP: Disease Activity Score (28 joints) using C-reactive protein; MMP-3: matrix metalloproteinase-3; NS: not significant at 5%; SAA: serum amyloid A. In FIG. 7B, LS mean differences in changes from baseline between sarilumab and adalimumab at week 24 by baseline biomarker tertiles in HAQ-DI (Top) and Pain VAS (Bottom). *Nominal biomarker-by-treatment interaction vs low tertile. Low, medium and high subgroups are based on biomarker tertile values in overall treatment groups (see Table 1 for tertile ranges). CI: confidence interval; CXCL13: chemokine (C—X—C motif) ligand 13; HAQ-DI: Health Assessment Questionnaire-Disability Index; LS: least squares; MMP-3: matrix metalloproteinase-3; NS: not significant at 5%; PINP: procollagen type 1 N-terminal propeptide; SAA: serum amyloid A; VAS: visual analogue scale.

FIG. 8 depicts ACR50 responses at week 24 and corresponding ORs with differential combinations of CXCL13 and sICAM-1. *Nominal P<0.05. ORs presented with 95% CIs. ACR50: American College of Rheumatology 50% improvement criteria; CI: confidence interval; CXCL13: chemokine (C—X—C motif) ligand 13; OR: odds ratio; q2w: every 2 weeks; sICAM-1: soluble intercellular adhesion molecule-1.

### DETAILED DESCRIPTION

This disclosure provides evidence that several biomarkers, including MMP-3, SAA, hepcidin, CXCL13, SICAM-1, and CRP, were associated with clinical efficacy and individually predicted response to sarilumab treatment. For example, patients with the highest baseline concentrations of SAA who received sarilumab were more likely to achieve ACR20/50/70 or DAS28-CRP <3.2 responses than with adalimumab compared with patients in the low tertile: ACR20 (OR [95% CI] 5.5 [2.1, 14.5]); ACR50 (5.4 [2.2, 13.2]); ACR70 (5.7 [1.8, 18.4]); DAS28-CRP <3.2 (6.1 [2.3, 15.7]). SAA was consistently predictive compared with high MMP-3 and CRP, which were only predictive of ACR20 and DAS28-CRP <3.2 response (Table 5). Baseline levels of biomarkers associated with bone remodeling, synovial lymphoid and myeloid cell infiltrates and anemia of inflammation were not predictive of efficacy at week 24, except for hepcidin and CXCL13, which were associated with ACR20 response.

Analysis of the pharmacodynamic effects of treatment on circulating biomarkers found sarilumab treatment reduced biomarkers of the acute-phase response, bone resorption, synovial inflammation and CV risk compared with adalimumab. These effects were generally observed early and persisted through to week 24. This was particularly evident with CRP and is consistent with previous observations. In addition, a greater proportion of patients treated with sarilumab vs adalimumab monotherapy demonstrated normalization of serum biomarkers at week 24, which was greatest for biomarkers of the acute-phase response (CRP and SAA).

As provided herein, both SAA and CRP were strongly correlated with DAS28-CRP at baseline (nominal P<0.0001). However, no correlations were observed between baseline biomarkers and PROs. Reductions in several biomarkers were associated with clinical efficacy at week 24 in adalimumab-treated patients; however, these associations were not observed in the sarilumab group. This result suggests that IL-6 receptor blockade may have a direct effect on production of these biomarkers independent of its effects on disease activity, in contrast to TNF inhibitors.

High levels of CRP, SAA, MMP-3, CXCL13 and sICAM-1 predicted ACR20 responses to sarilumab. These markers were associated with changes in several patients reported outcomes (PROs). High baseline levels of SAA and MMP-3 were also associated with improvements in several PROs, including patient global VAS, HAQ-DI, pain VAS, SF-36 PCS and MCS scores, morning stiffness VAS and RAID score.

### C-Reactive Protein (CRP) (GenBank Reference: NP 001315986.1)

The protein encoded by this gene belongs to the pentaxin family. It is involved in several host defense related functions based on its ability to recognize foreign pathogens and damaged cells of the host and to initiate their elimination by interacting with humoral and cellular effector systems in the blood. Consequently, the level of this protein in plasma increases greatly during acute phase response to tissue injury, infection, or other inflammatory stimuli.

In certain embodiments, the serum concentration of CRP tends to range from 1.0-3.4 mg/l in low tertile patients, 6.9-13.1 mg/l in medium tertile patients and 27.9-65.1 mg/l in high tertile patients. In certain embodiments, patients whose serum CRP concentrations are in the high tertile can achieve ACR20, DAS28-CRP score of less than 3.2 or improvement in patient global VAS, HAQ-DI, Pain VAS, SF-36-PCS score or morning stiffness VAS after 24 weeks of treatment with an IL-6 receptor antibody. In certain embodiments, patients whose serum CRP are in the medium tertile can achieve an improvement in patient global VAS, HAQ-DI, Pain VAS, SF-36-PCS score, SF-36-PF domain, morning stiffness VAS or RAID score after 24 weeks of treatment with an IL-6 receptor antibody.

In certain embodiments, the nucleic acid and amino acid sequences of CRP are provided below.

CRP transcript variant 2 cDNA sequence
(GenBank reference: NP_001315986.1):
(SEQ ID NO: 11)
ATGGAGAAGCTGTTGTGTTTCTTGGTCTTGACCAGCCTCTCTCATGCTTTT
GGCCAGACAGACATGTCGAGGAAGGCTTTTGTGTTTCCCAAAGAGTCGGAT
ACTTCCTATGTATCCCTCAAAGCACCGTTAACGAAGCCTCTCAAAGCCTTC
ACTGTGTGCCTCCACTTCTACACGGAACTGTCCTCGACCCGTGGGTACAGT
ATTTTCTCGTATGCCACCAAGAGACAAGACAATGAGATTCTCATATTTTGG
TCTAAGGATATAGGATACAGTTTTACAGTGGGTGGGTCTGAAATATTATTC
GAGGTTCCTGAAGTCACAGTAGCTCCAGTACACATTTGTACAAGCTGGGAG
TCCGCCTCAGGGATCGTGGAGTTCTGGGTAGATGGGAAGCCCAGGGTGAGG
AAGAGTCTGAAGAAGGGATACACTGTGGGGGCAGAAGCAAGCATCATCTTG
GGGCAGGAGCAGGATTCCTTCGGTGGGAACTTTGAAGGAAGCCAGTCCCTG
GTGGGAGACATTGGAAATGTGAACATGTGGGACTTTGTGCTGTCACCAGAT
GAGATTAACACCATCTATCTTGGCGGGCCCTTCAGTCCTAATGTCCTGAAC
TGGCGGGCACTGAAGTATGAAGTGCAAGGCGAAGTGTTCACCAAACCCCAG
CTGTGGCCCTGA
Note: CRP transcript variant 2 retains an intron
in the 3′ UTR compared to variant 1. Both variants
1 and 2 encode the same isoform.
CRP peptide sequence:
(SEQ ID NO: 12)
MEKLLCFLVLTSLSHAFGQTDMSRKAFVFPKESDTSYVSLKAPLTKPLKAF
TVCLHFYTELSSTRGYSIFSYATKRQDNEILIFWSKDIGYSFTVGGSEILF
EVPEVTVAPVHICTSWESASGIVEFWVDGKPRVRKSLKKGYTVGAEASIIL
GQEQDSFGGNFEGSQSLVGDIGNVNMWDFVLSPDEINTIYLGGPFSPNVLN
WRALKYEVQGEVFTKPQLWP
CRP genomic DNA sequence (GenBank reference:
NG_013007.1):
(SEQ ID NO: 13)
GACTGGATTCAGAGACTCAAACAATGTTATTGAGGCATGGTCTATCTCTCA
GCTCTACTCGTGAGTCAAGGATGGTGTATTAGTTGGTTTTCACACTGCTGT
AAAGAACTACCTGAGTATGGGTAATTTATAAACAAAAGAAATTTTAAATGA
ACTTACAGTTCCACATGTTTGGGGAGGACTCATGAAACTTACAATCATGGT
GGAAGGTGAAGGGGAAGCAGGCATTTTCTTCACAAGGCAGCAGGAGAGAGA
CAGTGTGAGTGGGGGACTGCCAAGCACTTTTATTTAAATCATCAGACCTAG
TGAGAACTCATTATCATGAGCACAGCATGGGCAAAACTACCTCCACGATCC
AATCTTCTCCCACCATGTCCCTCCCTCAACTCATGGGGATTACAATTTGAG
ATGACATTTGGGTGGGAACACAGAACCAAACCATATCATTCCACCTCTGGC
TCCTCCAAAATATCATGTTCTTTTCACATTTCAAAACCAATCATACCTTCC
CAACAGTCACCCAAACTTAACTCATTTCAGCATTAACTCAAAAGTCCAAGT
CTAAAGTTCCATCTGAGAAAAGGCAAGTCACTTCTGCCTATTAGCCTAGTA
AAATAAAAAACAAGTTAGTTACTTCCAAGATACAGTGGGGGTATAGGCATT
GGGTAAATGGTCCTGTTTGAAATGGGAGAAATTGGCCAAAACAAAGGGGCC
ACAGGCCCCATGTAAATCCAAAATCTGGCAGGACACTCATGAAATCTTAAA
GCTCCAAAATAATCTCCTTTGATTCTTTGTCTCACATCCAGGGCATGCTGA
TGCAAGCGGTAGGCTTCCATGGCCTTGGGTAGCTCCATACTTGTGGCTCTT
CAGGGTACAGCCCCTGTGGCTGCTTTCACAGGCTGGCATTGAACACTTGCA
AGCTTTTCTAAGCACAAGGTGCAAACTGTCAGTGGTTCTACCATTCTGGGA
TCTGGAGGACAGTGGCCCTCTTCTCACAGATCCACTAGGCAGTGCCCCAGT
GGGGACTCTGTGTGGAGACTCCAACCCCACATTTCCCTGCTGCATTGCCCT
AGTAGAGGTTTTCTGTGAGGGCTCCATGCCTGCAGAAGACTTCTGCCTGAA
CATCCAGGTGTTTCCATACATCTTCTGAAATCTAGACAGAAACTCCCAAAG
CTCAACTCTTGTCTTCTGTGCATCTGCACCCTCAACACTACTTGGAAGCCA
CCAAGGCTTGGGGCTTGTGCCCTCTGAAGCAATGGCCTGAGCTATATACAT
TGCCCCCTTTTAGCCATGGCTGGAGCCGCAGCAGCTGGCACACAGGGTGCC
ATGTTCCTGGGCTGCACAGAGCAGCGGGGCCCTGGGCCTGGCCCATGATAC
CATTTTTTCCTCCTAGGCTTTTGGACCTCTGATGGGAGGGGCTGCCATGAA
GATCTTCTGAAATGACCTGAAGACATTTTCCTCATTGTTTTGGCTATCAAC
ATTCATCTCCTCATTACTTATGCAAATTTCTGCAGCCAGCTTGAATTTTTC
CCCAGAAAATGGGTTTTTCTTTTCTACCACATGGTCAGGCTGCACATTTTC
CAAACTTTTATGCTCCCTTTCCCTTTTAAACATAAGTTCCAATTTCAGATC
ATCTCTTTGTGAACACATATGATTGTATGTTTTCAGAAAAAGCCAGGTCAC
TTCTTGAATGCTTTGGTGCTTAGAAATTTCTTAAGCACCAAAGCATTCAAG
AAATCATGTCTCTTAAGTTAAAAGTTCCACAGATCTCTAGGGCATGGGCAA
AATGCCACCATTGTCTTTGCTAAAACATAGAAAGAGTGACCTTTACTCCCG
TTTCCAATAAGTTCCTCATCTCCATCTAAGGACACCTCTGCATGAACTTCA
TTTTCCATATCACTATCAGCATTTTGGTCAAAACCATTCAACAAAACTCAG
GAAGTTCCAAGCTTTTCCACATCTTCCTGTCTTCTCCTGAGCCCTCCAAAC
TCTTCCAGCCTCTGCCCCTAGTTGGTTCCAA

### Matrix Metallopeptidase 3 (MMP-3) (Genbank Reference: EAW67032.1)

Proteins of the matrix metalloproteinase (MMP) family are involved in the breakdown of extracellular matrix in normal physiological processes, such as embryonic development, reproduction, and tissue remodeling, as well as in disease processes, such as arthritis and metastasis. Most MMP's are secreted as inactive proproteins which are activated when cleaved by extracellular proteinases. This gene encodes an enzyme which degrades fibronectin, laminin, collagens III, IV, IX, and X, and cartilage proteoglycans. The enzyme is thought to be involved in wound repair, progression of atherosclerosis, and tumor initiation. The gene is part of a cluster of MMP genes which localize to chromosome 11q22.3.

In certain embodiments, the serum concentration of MMP-3 tends to range from 10.3-20.8 ng/ml in low tertile patients, 35.5-54.1 ng/ml in medium tertile patients and 77.0-154.3 ng/ml in high tertile patients. In certain embodiments, patients whose serum MMP-3 concentrations are in the high tertile can achieve ACR20, DAS28-CRP score of less than 3.2 or an improvement in patient global VAS, HAQ-DI, Pain VAS, SF-36-PCS score or morning stiffness VAS after 24 weeks of treatment with an IL-6 receptor antibody. In certain embodiments, patients whose serum MMP-3 concentrations are in the medium tertile can achieve an improvement in pain VAS after 24 weeks of treatment with an IL-6 receptor antibody.

In certain embodiments, the nucleic acid and amino acid sequences of MMP-3 are provided below.

MMP-3 peptide sequence:
(SEQ ID NO: 14)
MKSLPILLLLCVAVCSAYPLDGAARGEDTSMNLVQKYLENYYDLKKDVKQF
VRRKDSGPVVKKIREMQKFLGLEVTGKLDSDTLEVMRKPRCGVPDVGHFRT
FPGIPKWRKTHLTYRIVNYTPDLPKDAVDSAVEKALKVWEEVTPLTFSRLY
EGEADIMISFAVREHGDFYPFDGPGNVLAHAYAPGPGINGDAHFDDDEQWT
KDTTGTNLFLVAAHEIGHSLGLFHSANTEALMYPLYHSLTDLTRFRLSQDD
INGIQSLYGPPPDSPETPLVPTEPVPPEPGTPANCDPALSFDAVSTLRGEI
LIFKDRHFWRKSLRKLEPELHLISSFWPSLPSGVDAAYEVTSKDLVFIFKG
NQFWAIRGNEVRAGYPRGIHTLGFPPTVRKIDAAISDKEKNKTYFFVEDKY
WRFDEKRNSMEPGFPKQIAEDFPGIDSKIDAVFEEFGFFYFFTGSSQLEFD
PNAKKVTHTLKSNSWLNC
MMP-3 cDNA sequence:
(SEQ ID NO: 15)
ATGAAGAGTCTTCCAATCCTACTGTTGCTGTGCGTGGCAGTTTGCTCAGCC
TATCCATTGGATGGAGCTGCAAGGGGTGAGGACACCAGCATGAACCTTGTT
CAGAAATATCTAGAAAACTACTACGACCTCAAAAAAGATGTGAAACAGTTT
GTTAGGAGAAAGGACAGTGGTCCTGTTGTTAAAAAAATCCGAGAAATGCAG
AAGTTCCTTGGATTGGAGGTGACGGGGAAGCTGGACTCCGACACTCTGGAG
GTGATGCGCAAGCCCAGGTGTGGAGTTCCTGATGTTGGTCACTTCAGAACC
TTTCCTGGCATCCCGAAGTGGAGGAAAACCCACCTTACATACAGGATTGTG
AATTATACACCAGATTTGCCAAAAGATGCTGTTGATTCTGCTGTTGAGAAA
GCTCTGAAAGTCTGGGAAGAGGTGACTCCACTCACATTCTCCAGGCTGTAT
GAAGGAGAGGCTGATATAATGATCTCTTTTGCAGTTAGAGAACATGGAGAC
TTTTACCCTTTTGATGGACCTGGAAATGTTTTGGCCCATGCCTATGCCCCT
GGGCCAGGGATTAATGGAGATGCCCACTTTGATGATGATGAACAATGGACA
AAGGATACAACAGGGACCAATTTATTTCTCGTTGCTGCTCATGAAATTGGC
CACTCCCTGGGTCTCTTTCACTCAGCCAACACTGAAGCTTTGATGTACCCA
CTCTATCACTCACTCACAGACCTGACTCGGTTCCGCCTGTCTCAAGATGAT
ATAAATGGCATTCAGTCCCTCTATGGACCTCCCCCTGACTCCCCTGAGACC
CCCCTGGTACCCACGGAACCTGTCCCTCCAGAACCTGGGACGCCAGCCAAC
TGTGATCCTGCTTTGTCCTTTGATGCTGTCAGCACTCTGAGGGGAGAAATC
CTGATCTTTAAAGACAGGCACTTTTGGCGCAAATCCCTCAGGAAGCTTGAA
CCTGAATTGCATTTGATCTCTTCATTTTGGCCATCTCTTCCTTCAGGCGTG
GATGCCGCATATGAAGTTACTAGCAAGGACCTCGTTTTCATTTTTAAAGGA
AATCAATTCTGGGCTATCAGAGGAAATGAGGTACGAGCTGGATACCCAAGA
GGCATCCACACCCTAGGTTTCCCTCCAACCGTGAGGAAAATCGATGCAGCC
ATTTCTGATAAGGAAAAGAACAAAACATATTTCTTTGTAGAGGACAAATAC
TGGAGATTTGATGAGAAGAGAAATTCCATGGAGCCAGGCTTTCCCAAGCAA
ATAGCTGAAGACTTTCCAGGGATTGACTCAAAGATTGATGCTGTTTTTGAA
GAATTTGGGTTCTTTTATTTCTTTACTGGATCTTCACAGTTGGAGTTTGAC
CCAAATGCAAAGAAAGTGACACACACTTTGAAGAGTAACAGCTGGCTTAAT
TGTTGA
MMP-3 mRNA sequence (GenBank reference:
NM_002422.5):
(SEQ ID NO: 16)
ACAAGGAGGCAGGCAAGACAGCAAGGCATAGAGACAACATAGAGCTAAGTA
AAGCCAGTGGAAATGAAGAGTCTTCCAATCCTACTGTTGCTGTGCGTGGCA
GTTTGCTCAGCCTATCCATTGGATGGAGCTGCAAGGGGTGAGGACACCAGC
ATGAACCTTGTTCAGAAATATCTAGAAAACTACTACGACCTCAAAAAAGAT
GTGAAACAGTTTGTTAGGAGAAAGGACAGTGGTCCTGTTGTTAAAAAAATC
CGAGAAATGCAGAAGTTCCTTGGATTGGAGGTGACGGGGAAGCTGGACTCC
GACACTCTGGAGGTGATGCGCAAGCCCAGGTGTGGAGTTCCTGATGTTGGT
CACTTCAGAACCTTTCCTGGCATCCCGAAGTGGAGGAAAACCCACCTTACA
TACAGGATTGTGAATTATACACCAGATTTGCCAAAAGATGCTGTTGATTCT
GCTGTTGAGAAAGCTCTGAAAGTCTGGGAAGAGGTGACTCCACTCACATTC
TCCAGGCTGTATGAAGGAGAGGCTGATATAATGATCTCTTTTGCAGTTAGA
GAACATGGAGACTTTTACCCTTTTGATGGACCTGGAAATGTTTTGGCCCAT
GCCTATGCCCCTGGGCCAGGGATTAATGGAGATGCCCACTTTGATGATGAT
GAACAATGGACAAAGGATACAACAGGGACCAATTTATTTCTCGTTGCTGCT
CATGAAATTGGCCACTCCCTGGGTCTCTTTCACTCAGCCAACACTGAAGCT
TTGATGTACCCACTCTATCACTCACTCACAGACCTGACTCGGTTCCGCCTG
TCTCAAGATGATATAAATGGCATTCAGTCCCTCTATGGACCTCCCCCTGAC
TCCCCTGAGACCCCCCTGGTACCCACGGAACCTGTCCCTCCAGAACCTGGG
ACGCCAGCCAACTGTGATCCTGCTTTGTCCTTTGATGCTGTCAGCACTCTG
AGGGGAGAAATCCTGATCTTTAAAGACAGGCACTTTTGGCGCAAATCCCTC
AGGAAGCTTGAACCTGAATTGCATTTGATCTCTTCATTTTGGCCATCTCTT
CCTTCAGGCGTGGATGCCGCATATGAAGTTACTAGCAAGGACCTCGTTTTC
ATTTTTAAAGGAAATCAATTCTGGGCTATCAGAGGAAATGAGGTACGAGCT
GGATACCCAAGAGGCATCCACACCCTAGGTTTCCCTCCAACCGTGAGGAAA
ATCGATGCAGCCATTTCTGATAAGGAAAAGAACAAAACATATTTCTTTGTA
GAGGACAAATACTGGAGATTTGATGAGAAGAGAAATTCCATGGAGCCAGGC
TTTCCCAAGCAAATAGCTGAAGACTTTCCAGGGATTGACTCAAAGATTGAT
GCTGTTTTTGAAGAATTTGGGTTCTTTTATTTCTTTACTGGATCTTCACAG
TTGGAGTTTGACCCAAATGCAAAGAAAGTGACACACACTTTGAAGAGTAAC
AGCTGGCTTAATTGTTGAAAGAGATATGTAGAAGGCACAATATGGGCACTT
TAAATGAAGCTAATAATTCTTCACCTAAGTCTCTGTGAATTGAAATGTTCG
TTTTCTCCTGCCTGTGCTGTGACTCGAGTCACACTCAAGGGAACTTGAGCG
TGAATCTGTATCTTGCCGGTCATTTTTATGTTATTACAGGGCATTCAAATG
GGCTGCTGCTTAGCTTGCACCTTGTCACATAGAGTGATCTTTCCCAAGAGA
AGGGGAAGCACTCGTGTGCAACAGACAAGTGACTGTATCTGTGTAGACTAT
TTGCTTATTTAATAAAGACGATTTGTCAGTTATTTTA

C—X—C Motif Chemokine Ligand 13 (CXCL13) (GenBank reference: NP 006410.1)

B lymphocyte chemoattractant, independently cloned and named Angie, is an antimicrobial peptide and CXC chemokine strongly expressed in the follicles of the spleen, lymph nodes, and Peyer's patches. It preferentially promotes the migration of B lymphocytes (compared to T cells and macrophages), apparently by stimulating calcium influx into, and chemotaxis of, cells expressing Burkitt's lymphoma receptor 1 (BLR-1). It may therefore function in the homing of B lymphocytes to follicles.

In certain embodiments, the serum concentration of CXCL13 tends to range from 52.4-72.0 pg/ml in low tertile patients, 98.2-130.6 pg/ml in medium tertile patients and 180.8-323.9 pg/ml in high tertile patients.

In certain embodiments, patients whose serum CXCL13 concentrations in the high tertile can achieve ACR20 or an improvement in HAQ-DI, SF-36-PCS score or SF-36-PF domain after 24 weeks of treatment with an antibody.

In certain embodiments, the nucleic acid and amino acid sequences of CXCL13 are provided below.

CXCL13 peptide sequence (GenBank reference:
NP_006410.1):
(SEQ ID NO: 17)
MKFISTSLLLMLLVSSLSPVQGVLEVYYTSLRCRCVQESSVFIPRRFIDRI
QILPRGNGCPRKEIIVWKKNKSIVCVDPQAEWIQRMMEVLRKRSSSTLPVP
VFKRKIP
CXCL13 cDNA sequence (GenBank reference:
NP_006410.1):
(SEQ ID NO: 18)
ATGAAGTTCATCTCGACATCTCTGCTTCTCATGCTGCTGGTCAGCAGCCTC
TCTCCAGTCCAAGGTGTTCTGGAGGTCTATTACACAAGCTTGAGGTGTAGA
TGTGTCCAAGAGAGCTCAGTCTTTATCCCTAGACGCTTCATTGATCGAATT
CAAATCTTGCCCCGTGGGAATGGTTGTCCAAGAAAAGAAATCATAGTCTGG
AAGAAGAACAAGTCAATTGTGTGTGTGGACCCTCAAGCTGAATGGATACAA
AGAATGATGGAAGTATTGAGAAAAAGAAGTTCTTCAACTCTACCAGTTCCA
GTGTTTAAGAGAAAGATTCCCTGA
CXCL13 genomic DNA sequence (GenBank reference:
NM_006419.2):
(SEQ ID NO: 19)
GAGAAGATGTTTGAAAAAACTGACTCTGCTAATGAGCCTGGACTCAGAGCT
CAAGTCTGAACTCTACCTCCAGACAGAATGAAGTTCATCTCGACATCTCTG
CTTCTCATGCTGCTGGTCAGCAGCCTCTCTCCAGTCCAAGGTGTTCTGGAG
GTCTATTACACAAGCTTGAGGTGTAGATGTGTCCAAGAGAGCTCAGTCTTT
ATCCCTAGACGCTTCATTGATCGAATTCAAATCTTGCCCCGTGGGAATGGT
TGTCCAAGAAAAGAAATCATAGTCTGGAAGAAGAACAAGTCAATTGTGTGT
GTGGACCCTCAAGCTGAATGGATACAAAGAATGATGGAAGTATTGAGAAAA
AGAAGTTCTTCAACTCTACCAGTTCCAGTGTTTAAGAGAAAGATTCCCTGA
TGCTGATATTTCCACTAAGAACACCTGCATTCTTCCCTTATCCCTGCTCTG
GATTTTAGTTTTGTGCTTAGTTAAATCTTTTCCAGGAAAAAGAACTTCCCC
ATACAAATAAGCATGAGACTATGTAAAAATAACCTTGCAGAAGCTGATGGG
GCAAACTCAAGCTTCTTCACTCACAGCACCCTATATACACTTGGAGTTTGC
ATTCTTATTCATCAGGGAGGAAAGTTTCTTTGAAAATAGTTATTCAGTTAT
AAGTAATACAGGATTATTTTGATTATATACTTGTTGTTTAATGTTTAAAAT
TTCTTAGAAAACAATGGAATGAGAATTTAAGCCTCAAATTTGAACATGTGG
CTTGAATTAAGAAGAAAATTATGGCATATATTAAAAGCAGGCTTCTATGAA
AGACTCAAAAAGCTGCCTGGGAGGCAGATGGAACTTGAGCCTGTCAAGAGG
CAAAGGAATCCATGTAGTAGATATCCTCTGCTTAAAAACTCACTACGGAGG
AGAATTAAGTCCTACTTTTAAAGAATTTCTTTATAAAATTTACTGTCTAAG
ATTAATAGCATTCGAAGATCCCCAGACTTCATAGAATACTCAGGGAAAGCA
TTTAAAGGGTGATGTACACATGTATCCTTTCACACATTTGCCTTGACAAAC
TTCTTTCACTCACATCTTTTTCACTGACTTTTTTTGTGGGGGGCGGGGCCG
GGGGGACTCTGGTATCTAATTCTTTAATGATTCCTATAAATCTAATGACAT
TCAATAAAGTTGAGCAAACATTTTACTTAAAAAAAAAAAAAAAAAA

### Serum Amyloid a (SAA) (GenBank Reference: AAB24060.1)

This gene encodes a member of the serum amyloid A family of apolipoproteins. The encoded preproprotein is proteolytically processed to generate the mature protein. This protein is a major acute phase protein that is highly expressed in response to inflammation and tissue injury. This protein also plays an important role in HDL metabolism and cholesterol homeostasis. High levels of this protein are associated with chronic inflammatory diseases including atherosclerosis, rheumatoid arthritis, Alzheimer's disease and Crohn's disease. This protein may also be a potential biomarker for certain tumors. Alternate splicing results in multiple transcript variants that encode the same protein. A pseudogene of this gene is found on chromosome 11.

In certain embodiments, the serum concentration of SAA tends to range from 2192.7-5346.4 ng/l in low tertile patients, 11832.0-30082.0 ng/l in medium tertile patients and 105200.0-256000.0 ng/l in high tertile patients. In certain embodiments, patients whose serum SAA concentrations are in the high tertile can achieve ACR20, ACR50 or ACR70, DAS28-CRP score of less than 3.2, or an improvement in patient global VAS, HAQ-DI, Pain VAS, SF-36-PCS score, SF-36-PF domain, morning stiffness VAS or RAID score after 24 weeks of treatment with an antibody. In certain embodiments, patients whose serum SAA concentrations are in the medium tertile can achieve an improvement in HAQ-DI after 24 weeks of treatment with an antibody.

In certain embodiments, the nucleic acid and amino acid sequences of SAA are provided below.

Serum amyloid A peptide sequence (GenBank
reference: AAB24060.1):
(SEQ ID NO: 20)
MRLFTGIVFCSLVMGVTSESWRSFFKEALQGVGDMGRAYWDIMISNHQNSN
RYLYARGNYDAAQRGPGGVWAAKLISRSRVYLQGLIDYYLFGNSSTVLEDS
KSNEKAEEWGRSGKDPDRFRPDGLPKKY
Serum amyloid A cDNA sequence (GenBank reference:
AAB24060.1):
(SEQ ID NO: 21)
ATGAGGCTTTTCACAGGCATTGTTTTCTGCTCCTTGGTCATGGGAGTCACC
AGTGAAAGCTGGCGTTCGTTTTTCAAGGAGGCTCTCCAAGGGGTTGGGGAC
ATGGGCAGAGCCTATTGGGACATAATGATATCCAATCACCAAAATTCAAAC
AGATATCTCTATGCTCGGGGAAACTATGATGCTGCCCAAAGAGGACCTGGG
GGTGTCTGGGCTGCTAAACTCATCAGCCGTTCCAGGGTCTATCTTCAGGGA
TTAATAGACTACTATTTATTTGGAAACAGCAGCACTGTATTGGAGGACTCG
AAGTCCAACGAGAAAGCTGAGGAATGGGGCCGGAGTGGCAAAGACCCCGAC
CGCTTCAGACCTGACGGCCTGCCTAAGAAATACTGA
Serum amyloid A mRNA sequence (GenBank reference:
M81349.1):
(SEQ ID NO: 22)
TATAGCTCCACGGCCAGAAGATACCAGCAGCTCTGCCTTTACTGAAATTTC
AGCTGGAGAAAGGTCCACAGCACAATGAGGCTTTTCACAGGCATTGTTTTC
TGCTCCTTGGTCATGGGAGTCACCAGTGAAAGCTGGCGTTCGTTTTTCAAG
GAGGCTCTCCAAGGGGTTGGGGACATGGGCAGAGCCTATTGGGACATAATG
ATATCCAATCACCAAAATTCAAACAGATATCTCTATGCTCGGGGAAACTAT
GATGCTGCCCAAAGAGGACCTGGGGTGTCTGGGCTGCTAAACTCATCAGCC
GTTCCAGGGTCTATCTTCAGGGATTAATAGACTACTATTTATTTGGAAACA
GCAGCACTGTATTGGAGGACTCGAAGTCCAACGAGAAAGCTGAGGAATGGG
GCCGGAGTGGCAAAGACCCCGACCGCTTCAGACCTGACGGCCTGCCTAAGA
AATACTGAGCTTCCTGCTCCTCTGCTCTCAGGGAAACTGGGCTGTGAGCCA
CACACTTCTCCCCCCAGACAGGGACACAGGGTCACTGAGCTTTGTGTCCCC
AGGAACTGGTATAGGGCACCTAGAGGTGTTCAATAAATGTTTGTCAAATTG
A

Soluble Intercellular Adhesion Molecule-1 (sICAM-1) (GenBank Reference: NP_000192.2):

All ICAM proteins are type I transmembrane glycoproteins, contain 2-9 immunoglobulin-like C2-type domains, and bind to the leukocyte adhesion LFA-1 protein. This protein may play a role in lymphocyte recirculation by blocking LFA-1-dependent cell adhesion. It mediates adhesive interactions important for antigen-specific immune response, NK-cell mediated clearance, lymphocyte recirculation, and other cellular interactions important for immune response and surveillance. Several transcript variants encoding the same protein have been found for this gene.

The serum concentration of sICAM-1 tends to range from 179.7-212.1 ng/ml in low tertile patients, 239.7-272.3 ng/ml in medium tertile patients and 313.7-380.0 ng/ml in high tertile patients. In certain embodiments, patients whose serum CXCL13 concentration in the low tertile and whose sICAM-1 concentration is in the low tertile can achieve ACR50 after 24 weeks of treatment with an antibody. In certain embodiments, patients whose serum CXCL13 concentration in the high tertile and whose sICAM-1 concentration is in the high tertile can achieve ACR50 after 24 weeks of treatment with an antibody.

In certain embodiments, the nucleic acid and amino acid sequences of sICAM-1 are provided below.

sICAM-1 peptide sequence (GenBank reference:
NP_000192.2):
(SEQ ID NO: 23)
MAPSSPRPALPALLVLLGALFPGPGNAQTSVSPSKVILPRGGSVLVTCSTS
CDQPKLLGIETPLPKKELLLPGNNRKVYELSNVQEDSQPMCYSNCPDGQST
AKTFLTVYWTPERVELAPLPSWQPVGKNLTLRCQVEGGAPRANLTVVLLRG
EKELKREPAVGEPAEVTTTVLVRRDHHGANFSCRTELDLRPQGLELFENTS
APYQLQTFVLPATPPQLVSPRVLEVDTQGTVVCSLDGLFPVSEAQVHLALG
DQRLNPTVTYGNDSFSAKASVSVTAEDEGTQRLTCAVILGNQSQETLQTVT
IYSFPAPNVILTKPEVSEGTEVTVKCEAHPRAKVTLNGVPAQPLGPRAQLL
LKATPEDNGRSFSCSATLEVAGQLIHKNQTRELRVLYGPRLDERDCPGNWT
WPENSQQTPMCQAWGNPLPELKCLKDGTFPLPIGESVTVTRDLEGTYLCRA
RSTQGEVTRKVTVNVLSPRYEIVIITVVAAAVIMGTAGLSTYLYNRQRKIK
KYRLQQAQKGTPMKPNTQATPP
sICAM-1 cDNA sequence (GenBank reference:
NP_000192.2):
(SEQ ID NO: 24)
ATGGCTCCCAGCAGCCCCCGGCCCGCGCTGCCCGCACTCCTGGTCCTGCTC
GGGGCTCTGTTCCCAGGACCTGGCAATGCCCAGACATCTGTGTCCCCCTCA
AAAGTCATCCTGCCCCGGGGAGGCTCCGTGCTGGTGACTGCAGCACCTCCT
GTGACCAGCCCAAGTTGTTGGGCATAGAGACCCCGTTGCCTAAAAAGGAGT
TGCTCCTGCCTGGGAACAACCGGAAGGTGTATGAACTGAGCAATGTGCAAG
AAGATAGCCAACCAATGTGCTATTCAAACTGCCCTGATGGGCAGTCAACAG
CTAAAACCTTCCTCACCGTGTACTGGACTCCAGAACGGGTGGAACTGGCAC
CCCTCCCCTCTTGGCAGCCAGTGGGCAAGAACCTTACCCTACGCTGCCAGG
TGGAGGGTGGGGCACCCCGGGCCAACCTCACCGTGGTGCTGCTCCGTGGGG
AGAAGGAGCTGAAACGGGAGCCAGCTGTGGGGGAGCCCGCTGAGGTCACGA
CCACGGTGCTGGTGAGGAGAGATCACCATGGAGCCAATTTCTCGTGCCGCA
CTGAACTGGACCTGCGGCCCCAAGGGCTGGAGCTGTTTGAGAACACCTCGG
CCCCCTACCAGCTCCAGACCTTTGTCCTGCCAGCGACTCCCCCACAACTTG
TCAGCCCCCGGGTCCTAGAGGTGGACACGCAGGGGACCGTGGTCTGTTCCC
TGGACGGGCTGTTCCCAGTCTCGGAGGCCCAGGTCCACCTGGCACTGGGGG
ACCAGAGGTTGAACCCCACAGTCACCTATGGCAACGACTCCTTCTCGGCCA
AGGCCTCAGTCAGTGTGACCGCAGAGGACGAGGGCACCCAGCGGCTGACGT
GTGCAGTAATACTGGGGAACCAGAGCCAGGAGACACTGCAGACAGTGACCA
TCTACAGCTTTCCGGCGCCCAACGTGATTCTGACGAAGCCAGAGGTCTCAG
AAGGGACCGAGGTGACAGTGAAGTGTGAGGCCCACCCTAGAGCCAAGGTGA
CGCTGAATGGGGTTCCAGCCCAGCCACTGGGCCCGAGGGCCCAGCTCCTGC
TGAAGGCCACCCCAGAGGACAACGGGCGCAGCTTCTCCTGCTCTGCAACCC
TGGAGGTGGCCGGCCAGCTTATACACAAGAACCAGACCCGGGAGCTTCGTG
TCCTGTATGGCCCCCGACTGGACGAGAGGGATTGTCCGGGAAACTGGACGT
GGCCAGAAAATTCCCAGCAGACTCCAATGTGCCAGGCTTGGGGGAACCCAT
TGCCCGAGCTCAAGTGTCTAAAGGATGGCACTTTCCCACTGCCCATCGGGG
AATCAGTGACTGTCACTCGAGATCTTGAGGGCACCTACCTCTGTCGGGCCA
GGAGCACTCAAGGGGAGGTCACCCGCAAGGTGACCGTGAATGTGCTCTCCC
CCCGGTATGAGATTGTCATCATCACTGTGGTAGCAGCCGCAGTCATAATGG
GCACTGCAGGCCTCAGCACGTACCTCTATAACCGCCAGCGGAAGATCAAGA
AATACAGACTACAACAGGCCCAAAAAGGGACCCCCATGAAACCGAACACAC
AAGCCACGCCTCCCTGA
sICAM-1 mRNA sequence (GenBank reference:
NM_000201.3)
(SEQ ID NO: 25)
GAGCTCCTCTGCTACTCAGAGTTGCAACCTCAGCCTCGCTATGGCTCCCAG
CAGCCCCCGGCCCGCGCTGCCCGCACTCCTGGTCCTGCTCGGGGCTCTGTT
CCCAGGACCTGGCAATGCCCAGACATCTGTGTCCCCCTCAAAAGTCATCCT
GCCCCGGGGAGGCTCCGTGCTGGTGACATGCAGCACCTCCTGTGACCAGCC
CAAGTTGTTGGGCATAGAGACCCCGTTGCCTAAAAAGGAGTTGCTCCTGCC
TGGGAACAACCGGAAGGTGTATGAACTGAGCAATGTGCAAGAAGATAGCCA
ACCAATGTGCTATTCAAACTGCCCTGATGGGCAGTCAACAGCTAAAACCTT
CCTCACCGTGTACTGGACTCCAGAACGGGTGGAACTGGCACCCCTCCCCTC
TTGGCAGCCAGTGGGCAAGAACCTTACCCTACGCTGCCAGGTGGAGGGTGG
GGCACCCCGGGCCAACCTCACCGTGGTGCTGCTCCGTGGGGAGAAGGAGCT
GAAACGGGAGCCAGCTGTGGGGGAGCCCGCTGAGGTCACGACCACGGTGCT
GGTGAGGAGAGATCACCATGGAGCCAATTTCTCGTGCCGCACTGAACTGGA
CCTGCGGCCCCAAGGGCTGGAGCTGTTTGAGAACACCTCGGCCCCCTACCA
GCTCCAGACCTTTGTCCTGCCAGCGACTCCCCCACAACTTGTCAGCCCCCG
GGTCCTAGAGGTGGACACGCAGGGGACCGTGGTCTGTTCCCTGGACGGGCT
GTTCCCAGTCTCGGAGGCCCAGGTCCACCTGGCACTGGGGGACCAGAGGTT
GAACCCCACAGTCACCTATGGCAACGACTCCTTCTCGGCCAAGGCCTCAGT
CAGTGTGACCGCAGAGGACGAGGGCACCCAGCGGCTGACGTGTGCAGTAAT
ACTGGGGAACCAGAGCCAGGAGACACTGCAGACAGTGACCATCTACAGCTT
TCCGGCGCCCAACGTGATTCTGACGAAGCCAGAGGTCTCAGAAGGGACCGA
GGTGACAGTGAAGTGTGAGGCCCACCCTAGAGCCAAGGTGACGCTGAATGG
GGTTCCAGCCCAGCCACTGGGCCCGAGGGCCCAGCTCCTGCTGAAGGCCAC
CCCAGAGGACAACGGGCGCAGCTTCTCCTGCTCTGCAACCCTGGAGGTGGC
CGGCCAGCTTATACACAAGAACCAGACCCGGGAGCTTCGTGTCCTGTATGG
CCCCCGACTGGACGAGAGGGATTGTCCGGGAAACTGGACGTGGCCAGAAAA
TTCCCAGCAGACTCCAATGTGCCAGGCTTGGGGGAACCCATTGCCCGAGCT
CAAGTGTCTAAAGGATGGCACTTTCCCACTGCCCATCGGGGAATCAGTGAC
TGTCACTCGAGATCTTGAGGGCACCTACCTCTGTCGGGCCAGGAGCACTCA
AGGGGAGGTCACCCGCAAGGTGACCGTGAATGTGCTCTCCCCCCGGTATGA
GATTGTCATCATCACTGTGGTAGCAGCCGCAGTCATAATGGGCACTGCAGG
CCTCAGCACGTACCTCTATAACCGCCAGCGGAAGATCAAGAAATACAGACT
ACAACAGGCCCAAAAAGGGACCCCCATGAAACCGAACACACAAGCCACGCC
TCCCTGAACCTATCCCGGGACAGGGCCTCTTCCTCGGCCTTCCCATATTGG
TGGCAGTGGTGCCACACTGAACAGAGTGGAAGACATATGCCATGCAGCTAC
ACCTACCGGCCCTGGGACGCCGGAGGACAGGGCATTGTCCTCAGTCAGATA
CAACAGCATTTGGGGCCATGGTACCTGCACACCTAAAACACTAGGCCACGC
ATCTGATCTGTAGTCACATGACTAAGCCAAGAGGAAGGAGCAAGACTCAAG
ACATGATTGATGGATGTTAAAGTCTAGCCTGATGAGAGGGGAAGTGGTGGG
GGAGACATAGCCCCACCATGAGGACATACAACTGGGAAATACTGAAACTTG
CTGCCTATTGGGTATGCTGAGGCCCCACAGACTTACAGAAGAAGTGGCCCT
CCATAGACATGTGTAGCATCAAAACACAAAGGCCCACACTTCCTGACGGAT
GCCAGCTTGGGCACTGCTGTCTACTGACCCCAACCCTTGATGATATGTATT
TATTCATTTGTTATTTTACCAGCTATTTATTGAGTGTCTTTTATGTAGGCT
AAATGAACATAGGTCTCTGGCCTCACGGAGCTCCCAGTCCTAATCACATTC
AAGGTCACCAGGTACAGTTGTACAGGTTGTACACTGCAGGAGAGTGCCTGG
CAAAAAGATCAAATGGGGCTGGGACTTCTCATTGGCCAACCTGCCTTTCCC
CAGAAGGAGTGATTTTTCTATCGGCACAAAAGCACTATATGGACTGGTAAT
GGTTACAGGTTCAGAGATTACCCAGTGAGGCCTTATTCCTCCCTTCCCCCC
AAAACTGACACCTTTGTTAGCCACCTCCCCACCCACATACATTTCTGCCAG
TGTTCACAATGACACTCAGCGGTCATGTCTGGACATGAGTGCCCAGGGAAT
ATGCCCAAGCTATGCCTTGTCCTCTTGTCCTGTTTGCATTTCACTGGGAGC
TTGCACTATGCAGCTCCAGTTTCCTGCAGTGATCAGGGTCCTGCAAGCAGT
GGGGAAGGGGGCCAAGGTATTGGAGGACTCCCTCCCAGCTTTGGAAGCCTC
ATCCGCGTGTGTGTGTGTGTGTATGTGTAGACAAGCTCTCGCTCTGTCACC
CAGGCTGGAGTGCAGTGGTGCAATCATGGTTCACTGCAGTCTTGACCTTTT
GGGCTCAAGTGATCCTCCCACCTCAGCCTCCTGAGTAGCTGGGACCATAGG
CTCACAACACCACACCTGGCAAATTTGATTTTTTTTTTTTTTCCAGAGACG
GGGTCTCGCAACATTGCCCAGACTTCCTTTGTGTTAGTTAATAAAGCTTTC
TCAACTGCC

### Pain VAS

The visual analog scale (VAS) for pain is a unidimensional measure of pain intensity, which has been widely used in diverse adult populations, including those with rheumatic diseases. The pain VAS is a continuous scale comprised of a horizontal (HVAS) or vertical (VVAS) line, usually 10 centimeters (100 mm) in length, anchored by 2 verbal descriptors, one for each symptom extreme. For pain intensity, the scale is most commonly anchored by “no pain” (score of 0) and “pain as bad as it could be” or “worst imaginable pain” (score of 100 [100-mm scale]). To avoid clustering of scores around a preferred numeric value, numbers or verbal descriptors at intermediate points are not recommended.

### Patient Global VAS

The patient global assessment of disease activity is a crucial component of various measures of disease activity in rheumatoid arthritis (RA). Our objective was to identify underlying latent traits driving the patient global assessment using a quantitative, multivariable data reduction approach. In certain embodiments, patients rate their global assessment on a visual analog scale (VAS) by answering the question, “Considering all of the ways your disease affects you, how well are you doing in the past week?” Hence, the patient global not only assesses disease from the patient perspective but also encompasses various factors affecting patients in addition to RA.

### Morning Stiffness VAS

Patients were asked if there was joint stiffness upon waking (yes or no); those answering yes were asked to indicate the severity of the morning stiffness by marking a vertical line across the severity scale (100-mm visual analog scale [VAS], where 0=not severe at all and 100=extremely severe). These patients were also asked to record the time that morning stiffness in the joints subsided; the duration of morning stiffness was calculated by subtraction of this time from the waking time. Patients were also asked to record the intensity of pain upon waking, by marking a line on a 100-mm VAS (where 0=no pain and 100=very severe pain).

### Anti-IL-6R Antibodies

The present disclosure includes methods that comprise administering to a patient an antibody, or an antigen-binding fragment thereof, that binds specifically to hIL-6R. As used herein, the term “hIL-6R” means a human cytokine receptor that specifically binds human interleukin-6 (IL-6). In certain embodiments, the antibody that is administered to the patient binds specifically to the extracellular domain of hIL-6R.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, and bivalent nanobodies), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) VH-CH1; (ii) VH-CH2; (iii) VH-CH3; (iv) VH—CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may in various embodiments consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may in various embodiments comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may in various embodiments be adapted for use in the context of an antigen-binding fragment of an anti-IL-6R antibody using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies featured in the disclosure may in various embodiments nonetheless include amino acid residues not encoded by human germline immunoglobulin sequences (e.g., mutations introduced by random or site-specific mutagenesis in vitro or by somatic mutation in vivo), for example in the CDRs and in in some embodiments CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295, incorporated herein by reference in its entirety,) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In an embodiment, an immunoglobulin molecule comprises a stable four chain construct of approximately 150-160 kDa in which the dimers are held together by an interchain heavy chain disulfide bond. In certain embodiments, the dimers are not linked via inter-chain disulfide bonds and a molecule of about 75-80 kDa is formed composed of a covalently coupled light and heavy chain (half-antibody). These embodiments/forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al., (1993) Molecular Immunology 30:105, incorporated by reference in its entirety) to levels typically observed using a human IgG1 hinge. The instant disclosure encompasses in various embodiments antibodies having one or more mutations in the hinge, CH2 or CH3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” In various embodiments, the isolated antibody also includes an antibody in situ within a recombinant cell. In other embodiments, isolated antibodies are antibodies that have been subjected to at least one purification or isolation step. In various embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-6R, as used herein, includes antibodies that bind IL-6R or portion thereof with a KD of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or about 0.5 nM, as measured in a surface plasmon resonance assay. An isolated antibody that specifically binds human IL-6R may, however, have cross-reactivity to other antigens, such as IL-6R molecules from other (non-human) species.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “KD”, as used herein, is intended to refer to the equilibrium dissociation constant of an antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The anti-IL-6R antibodies useful for the methods featured herein may in various embodiments include one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes in various embodiments methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). Numerous antibodies and antigen-binding fragments may be constructed which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a certain germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-IL-6R antibodies comprising variants of any of the HCVR, LCVR, and/or CDR amino acid sequences disclosed herein having one or more conservative substitutions. For example, the present disclosure includes the use of anti-IL-6R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

According to the present disclosure, the anti-IL-6R antibody, or antigen-binding fragment thereof, in various embodiments comprises a heavy chain variable region (HCVR), light chain variable region (LCVR), and/or complementarity determining regions (CDRs) comprising any of the amino acid sequences of the anti-IL-6R antibodies as claimed in U.S. Pat. No. 7,582,298, incorporated herein by reference in its entirety. The anti-IL-6R antibody or antigen-binding fragment thereof that can be used in the context of the methods of the present disclosure comprises the heavy chain complementarity determining regions (HCDRs) of a HCVR comprising the amino acid sequence of SEQ ID NO: 1 and the light chain complementarity determining regions (LCDRs) of a LCVR comprising the amino acid sequence of SEQ ID NO:2. According to certain embodiments, the anti-IL-6R antibody or antigen-binding fragment thereof comprises three HCDRs (i.e., HCDR1, HCDR2 and HCDR3) and three LCDRs (i.e., LCDR1, LCDR2 and LCDR3), wherein the HCDR1 comprises the amino acid sequence of SEQ ID NO:3; the HCDR2 comprises the amino acid sequence of SEQ ID NO:4; the HCDR3 comprises the amino acid sequence of SEQ ID NO:5; the LCDR1 comprises the amino acid sequence of SEQ ID NO: 6; the LCDR2 comprises the amino acid sequence of SEQ ID NO:7; and the LCDR3 comprises the amino acid sequence of SEQ ID NO:8. In yet other embodiments, the anti-IL-6R antibody or antigen-binding fragment thereof comprises an HCVR comprising SEQ ID NO: 1 and an LCVR comprising SEQ ID NO:2.

In certain embodiments, the anti-IL-6R antibody or antigen-binding fragment thereof comprises a heavy chain comprising SEQ ID NO:9 and a light chain comprising SEQ ID NO:10. According to certain exemplary embodiments, the methods of the present disclosure comprise the use of the anti-IL-6R antibody referred to and known in the art as sarilumab, or a bioequivalent thereof.

The amino acid sequence of SEQ ID NO: 1 is
EVQLVESGGGLVQPGRSLRLSCAASRFTFDDYAMHWVRQAPGKGLEWVSGI
SWNSGRIGYADSVKGRFTISRDNAENSLFLQMNGLRAEDTALYYCAKGRDS
FDIWGQGTMVTVSS
The amino acid sequence of SEQ ID NO: 2 is
DIQMTQSPSSVSASVGDRVTITCRASQGISSWLAWYQQKPGKAPKLLIYGA
SSLESGVPSRFSGSGSGTDFTLTISSLQPEDFASYYCQQANSFPYTFGQGT
KLEIK.
The amino acid sequence of SEQ ID NO: 3 is
RFTFDDYA.
The amino acid sequence of SEQ ID NO: 4 is
ISWNSGRI.
The amino acid sequence of SEQ ID NO: 5 is
AKGRDSFDI.
The amino acid sequence of SEQ ID NO: 6 is
QGISSW.
The amino acid sequence of SEQ ID NO: 7 is
GAS.
The amino acid sequence of SEQ ID NO: 8 is
QQANSFPYT.
The amino acid sequence of SEQ ID NO: 9 is
EVQLVESGGGLVQPGRSLRLSCAASRFTFDDYAMHWVRQAPGKGLEWVSGI
SWNSGRIGYADSVKGRFTISRDNAENSLFLQMNGLRAEDTALYYCAKGRDS
FDIWGQGTMVTVSSASTKGPSVFPLAPSSKSTSGGTAALGCLVKDYFPEPV
TVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSK
LTVDKSRWQQGNVFSCSVMHEALHNHYTQKSLSLSPGK.
The amino acid sequence of SEQ ID NO: 10 is
DIQMTQSPSSVSASVGDRVTITCRASQGISSWLAWYQQKPGKAPKLLIYGA
SSLESGVPSRFSGSGSGTDFTLTISSLQPEDFASYYCQQANSFPYTFGQGT
KLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC.

The term “bioequivalent” as used herein, refers to a molecule having similar bioavailability (rate and extent of availability) after administration at the same molar dose and under similar conditions (e.g., same route of administration), such that the effect, with respect to both efficacy and safety, can be expected to be essentially same as the comparator molecule. Two pharmaceutical compositions comprising an anti-IL-6R antibody are bioequivalent if they are pharmaceutically equivalent, meaning they contain the same amount of active ingredient (e.g., IL-6R antibody), in the same dosage form, for the same route of administration and meeting the same or comparable standards. Bioequivalence can be determined, for example, by an in vivo study comparing a pharmacokinetic parameter for the two compositions. Parameters commonly used in bioequivalence studies include peak plasma concentration (Cmax) and area under the plasma drug concentration time curve (AUC).

The disclosure in certain embodiments relates to methods comprising administering to the subject an antibody which comprises the heavy chain variable region comprising sequence SEQ ID NO: 1 and the light chain variable region comprising sequence SEQ ID NO:2.

The disclosure provides pharmaceutical compositions comprising such antibody, and methods of using these compositions.

The antibody which comprises the heavy chain variable region comprising sequence SEQ ID NO: 1 and the light chain variable region comprising sequence SEQ ID NO:2 is an antibody that specifically binds human interleukin-6 receptor (hIL-6R). See international publication number WO2007/143168, incorporated herein by reference in its entirety.

In certain embodiments, the antibody which comprises the heavy chain variable region comprising sequence SEQ ID NO:1 and the light chain variable region comprising sequence SEQ ID NO:2 is sarilumab.

### DMARDs

DMARDs are drugs defined by their use in rheumatoid arthritis to slow down disease progression.

DMARDs have been classified as synthetic (sDMARD) and biological (bDMARD). Synthetic DMARDs include non-exhaustively methotrexate, sulfasalazine, leflunomide, and hydroxychloroquine. Biological DMARDs include non-exhaustively adalimumab, golimumab, etanercept, abatacept, infliximab, rituximab, and tocilizumab.

### Methods of Administration and Formulations

The methods described herein comprise administering to a patient a therapeutically effective amount of an anti-hIL-6R antibody alone and, optionally, a therapeutically effective amount of an anti-hIL-6R antibody in combination with a DMARD. As used herein, the phrase “therapeutically effective amount” means a dose of the therapeutic that results in a detectable improvement in one or more symptoms associated with depression or a depressive disorder or which causes a biological effect (e.g., a decrease in the level of a particular biomarker) that is correlated with the underlying pathologic mechanism(s) giving rise to the condition or symptom(s) of depression or a depressive disorder. For example, a dose of anti-hIL-6R antibody which causes an improvement in any of the following symptoms or conditions is deemed a “therapeutically effective amount”: loss of confidence and self-esteem, inappropriate guilt, thoughts of death and suicide, diminished concentration, disturbance of sleep and appetite and feelings of sadness and loss of interest across most situations.

The antibody in various embodiments is administered to the subject. In various embodiments, the antibody is administered once every two weeks. “Once every two weeks” has the same meaning as “q2w” or “once per two weeks”, i.e. that the antibody is administered once in a two week period of time. According to certain embodiments, the antibody is administered subcutaneously.

In certain embodiments, the antibody is administered at about 150 mg or about 200 mg once every two weeks. In this context, “about” refers to an amount within 5% of the stated amount. For example, “about 150 mg” is a range of between 142 and 158 mg, and “about 200 mg” is a range of between 90 and 210 mg. According to certain embodiments, the antibody is administered subcutaneously.

The antibody is administered to the subject in various embodiments in a formulation comprising suitable carriers, excipients, and other agents to provide improved transfer, delivery, tolerance, and the like, and suitable for a subcutaneous injection.

The injectable preparations may be prepared by methods publicly known. For example, injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 20 or 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injectable preparation thus prepared can be filled in an appropriate ampoule.

The antibody is typically formulated as described herein and in international publication number WO2011/085158, incorporated herein by reference in its entirety.

In various embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
between about 100 mg/mL and about 200 mg/mL of the antibody.

In certain embodiments, the antibody is administered as an aqueous buffered solution at pH 6.0 containing
about 21 mM histidine,
about 45 mM arginine,
about 0.2% (w/v) polysorbate 20,
about 5% (w/v) sucrose, and
at least about 130 mg/mL of the antibody.

In certain embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose, and
about 131.6 mg/mL of the antibody.

In certain embodiments, the antibody is administered as an aqueous buffered solution at about pH 6.0 containing
about 21 mM histidine,
about 45 mM arginine,
about 0.2% (w/v) polysorbate 20,
about 5% (w/v) sucrose; and
about 175 mg/mL of the antibody.

In certain embodiments, the antibody is administered as an aqueous buffered solution at pH 6.0 containing
21 mM histidine,
45 mM arginine,
0.2% (w/v) polysorbate 20,
5% (w/v) sucrose, and
between 100 mg/mL and 200 mg/mL of the antibody.

In certain embodiments, the antibody is administered as an aqueous buffered solution at pH 6.0 containing
21 mM histidine,
45 mM arginine,
0.2% (w/v) polysorbate 20,
5% (w/v) sucrose, and
at least 130 mg/mL of the antibody.

In certain embodiments, the antibody is administered as an aqueous buffered solution at pH 6.0 containing
21 mM histidine,
45 mM arginine,
0.2% (w/v) polysorbate 20,
5% (w/v) sucrose, and
131.6 mg/mL of the antibody.

In certain embodiments, the antibody is administered as an aqueous buffered solution at pH 6.0 containing
21 mM histidine,
45 mM arginine,
0.2% (w/v) polysorbate 20,
5% (w/v) sucrose; and
175 mg/mL of the antibody.

The antibody according to the disclosure can be administered to the subject using any acceptable device or mechanism. For example, the administration can be accomplished using a syringe and needle or with a reusable pen and/or autoinjector delivery device. The methods of the present disclosure include the use of numerous reusable pen and/or autoinjector delivery devices to administer an antibody (or pharmaceutical formulation comprising the antibody). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen and/or autoinjector delivery devices having applications in subcutaneous delivery of a pharmaceutical composition described herein include, but are not limited to, the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), the HUMIRA™ Pen (Abbott Labs, Abbott Park, IL), the DAI® Auto Injector (SHL Group) and any auto-injector featuring the PUSHCLICK™ technology (SHL Group), to name only a few.

In certain embodiments, the antibody is administered with a prefilled syringe.

In certain embodiments, the antibody is administered with a prefilled syringe containing a safety system. For example, the safety system prevents an accidental needstick injury. In various embodiments, the antibody is administered with a prefilled syringe containing an ERIS™ safety system (West Pharmaceutical Services Inc.). See also U.S. Pat. Nos. 5,215,534 and 9,248,242, incorporated herein by reference in their entireties.

In certain embodiments, the antibody is administered with an auto-injector. In various embodiments, the antibody is administered with an auto-injector featuring the PUSHCLICK™ technology (SHL Group). In various embodiments, the auto-injector is a device comprising a syringe that allows for administration of a dose of the composition and/or antibody to a subject. See also U.S. Pat. Nos. 9,427,531 and 9,566,395, incorporated herein by reference in their entireties.

### Patient Population

According to the disclosure, “subject” means a human subject or human patient.

The antibody according to the disclosure is in various embodiments administered to subjects who have serum biomarker concentrations as described above.

According to the disclosure, a subject who is considered “ineffectively treated” by his or her physician is a subject who in various embodiments either has shown to be intolerant to the one or more DMARD tested by the physician, and/or a subject who has shown an inadequate response to the one or more DMARD tested by the physician, typically a subject who is still considered by the physician to present with, or to have, active rheumatoid arthritis despite the previous one or more DMARD administered. The “active rheumatoid arthritis” is typically defined as:
at least 6 of 66 swollen joints and 8 of 68 tender joints, as counted by the physician in a typical quantitative swollen and tender joint count examination,
High sensitivity C-reactive protein (hs-CRP)≥8 mg/L or ESR≥28 mm/H
DAS28ESR>5.1.

In certain embodiments, “moderately active RA” in a subject is defined as: at least 8 and at most 26 of 68 tender joints and at least 6 and/or at most 16 of 66 swollen joints in the subject. In certain embodiments, “severely active RA” in a subject is defined as: (i) more than 27 of 68 tender joints and/or at least 17 of 66 swollen joints in the subject.

In some embodiments, the subject, who was previously ineffectively treated for rheumatoid arthritis by administering at least one DMARD different from the antibody, is a subject who was previously ineffectively treated for rheumatoid arthritis by administering a DMARD. In some embodiments, the DMARD is selected from the group consisting of methotrexate, sulfasalazine, leflunomide, and hydroxychloroquine. In various embodiments, the DMARD is methotrexate.

In some embodiments, the subject, who was previously ineffectively treated for rheumatoid arthritis by administering one or more DMARD different from the antibody, is a subject who had an inadequate response or intolerance to methotrexate.

According to the disclosure, for those subjects previously ineffectively treated for rheumatoid arthritis by administering one or more DMARD different from the antibody, the one or more DMARD is/are not administered anymore to the subject, and the antibody is in various embodiments administered alone, in monotherapy to the subject. See international publication number WO2017155990, which is incorporated by reference herein in its entirety.

In some embodiments, the subject is intolerant to the DMARD due to one or more physical reactions, conditions or symptoms from the treatment with the DMARD. Physical reactions, conditions or symptoms can include allergies, pain, nausea, diarrhea, azotemia, bleeding of the stomach, intestinal bleeding, canker sores, decreased blood platelets, perforation of the intestine, bacterial infection, inflammation of gums or mouth, inflammation of the stomach lining or intestinal lining, bacterial sepsis, stomach ulcer, intestinal ulcer, sun sensitive skin, dizziness, loss of appetite, low energy, and vomiting. In certain embodiments, intolerance can be determined by the subject or by a medical professional upon examination of the subject. In various embodiments, the DMARD is selected from the group consisting of methotrexate, sulfasalazine, leflunomide, and hydroxychloroquine. In some embodiments, the DMARD is methotrexate.

In some embodiments the disclosure comprises administering to the subject one or more additional therapeutic agents in combination with the IL-6R antibody. As used herein, the expression “in combination with” means that the additional therapeutic agents are administered before, after, or concurrent with the pharmaceutical composition comprising the IL-6R antibody. In certain embodiments, the subject is administered the antibody with a DMARD and/or TNF-α antagonist.

All publications mentioned herein are incorporated herein by reference in their entirety for all purposes.

### EXAMPLES

### Example 1—Sarilumab and Adalimumab Differential Effects on Bone Resorption and Cardiovascular Risk Biomarkers, and Predictions of Treatment Outcomes

### Methods

This phase III active-comparator randomized controlled trial has been described in full previously (Burmester G R, Ann Rheum Dis 2017; 76:840-7.) In brief, MTX-INT/IR patients were randomized to sarilumab 200 mg every 2 weeks (q2w) or adalimumab 40 mg q2w for 24 weeks. At week 16, dose escalation to weekly adalimumab was permitted for those who did not achieve ≥20% improvement in tender and swollen joint counts. The trial was conducted in accordance with Good Clinical Practice and with the principles of the Declaration of Helsinki; all protocols and patient information materials were approved by appropriate ethical review boards and all patients provided written informed consent.

### Efficacy and PRO Endpoints

Efficacy endpoints included: proportion of patients achieving ≥20/50/70% improvement according to American College of Rheumatology criteria (ACR20/50/70), Clinical Disease Activity Index (CDAI)≤2.8, CDAI≤10, DAS28 using CRP (DAS28-CRP) or DAS28-ESR<2.6 and DAS28-CRP or DAS28-ESR<3.2.

PROs evaluated in the study were previously described for the overall ITT population (Strand V, Arthritis Res Ther 2018; 20:129) and (evaluated as change from baseline at week 24) included Patient Global Assessment of disease activity visual analogue scale (VAS), Health Assessment Questionnaire-Disability Index (HAQ-DI), pain VAS, Functional Assessment of Chronic Illness Therapy (FACIT)-Fatigue, morning stiffness VAS, rheumatoid arthritis impact of disease (RAID) score and Medical Outcomes Study Short-Form (36-item) Health Survey (SF-36) physical (PCS) and mental (MCS) component summary scores, which include the physical functioning, role-physical, bodily pain, general health, vitality, social functioning, role-emotional and mental health domains.

### Serum Collection and Biomarker Analysis

Patients were selected for this biomarker analysis if they had been randomized and treated with sarilumab or adalimumab during the double-blind period, had provided written informed consent for future use of samples, with a serum sample drawn pre-dose (baseline) and evaluable (biomarker population). Serum samples were collected and stored frozen at baseline and post-treatment through week 24 from 307 patients in the intention-to-treat population (sarilumab: n=153; adalimumab: n=154). Baseline results are provided in Table 1.

TABLE 1
Baseline biomarker serum concentrations in the biomarker population

| | Adalimumab 40 mg q2w | Sarilumab 200 mg q2w | |
|---|---|---|---|
| Biomarker | (n = 154) | (n = 153) | Low tertile |
| SAA, ng/l | 22 806.0 (5817.2, 115 200.0) | 16 089.0 (4997.5, 85 918.0) | 3734.7 (2192.7, 5346.4) |
| CRP, mg/l | 9.4 (3.8, 33.5) | 7.8 (2.8, 24.7) | 1.9 (1.0, 3.4) |
| Lp(a), mg/l | 235.5 (111.0, 559.0) | 179.0 (78.0, 402.0)* | 48.5 (17.5, 100.0) |
| MMP-3, ng/ml | 44.0 (25.2, 80.9) | 40.8 (19.3, 74.4) | 16.0 (10.3, 20.8) |
| Total RANKL, pmol/l | 484.5 (254.7, 1423.1) | 547.6 (268.5, 1361.3) | 200.1 (136.7, 258.5) |
| P1NP, ng/ml | 45.9 (34.9, 63.9) | 44.7 (30.3, 59.9) | 27.6 (21.4, 32.9) |
| OPG, pmol/1 | 5.9 (5.0, 8.0) | 6.0 (4.7, 7.5) | 4.3 (3.9, 5.0) |
| OC, ng/ml | 19.0 (13.8, 26.0) | 18.0 (13.9, 25.8) | 12.0 (9.6, 13.8) |
| CXCL13, pg/ml | 120.1 (72.4, 184.7) | 112.8 (70.8, 180.8) | 61.8 (52.4, 72.0) |
| sICAM-1, ng/ml | 258.6 (212.1, 324.8) | 257.3 (212.7, 304.0) | 199.3 (179.7, 212.1) |
| Iron μmol/l | 10.5 (7.0, 14.9) | 11.3 (7.2, 16.0) | 6.1 (4.2, 7.0) |
| Ferritin, ng/ml | 80.0 (41.1, 174.0) | 74.9 (35.1, 130.6) | 24.9 (13.9, 35.5) |
| TIBC, μg/dl | 321.5 (293.5, 350.5) | 324.0 (303.0, 361.0) | 286.0 (267.0, 297.0) |
| Hepcidin, ng/ml | 24.8 (9.7, 48.9) | 20.9 (9.2, 39.3) | 6.0 (3.7, 9.3) |

| | | | Reference |
|---|---|---|---|
| Biomarker | Medium tertile | High tertile | range^{a} |
| SAA, ng/l | 18549.5 (11 832.0, 30 082.0) | 174 900.0 (105 200.0, 256 000.0) | 1000.0-9249.3 |
| CRP, mg/l | 8.5 (6.9, 13.1) | 37.6 (27.9, 65.1) | ≤2.9 |
| Lp(a), mg/l | 192.0 (157.0, 236.0) | 689.5 (450.0, 1116.0) | 19.0-1028.0 |
| MMP-3, ng/ml | 42.8 (35.5, 54.1) | 99.9 (77.0, 154.3) | 6.0-15.8 |
| Total RANKL, pmol/l | 515.0 (424.3, 674.0) | 2252.8 (1417.6, 3657.6) | 35.1-639.7 |
| P1NP, ng/ml | 45.6 (41.6, 50.1) | 73.2 (63.0, 87.6) | 47.9-204.1 |
| OPG, pmol/1 | 5.9 (5.6, 6.5) | 8.8 (7.7, 10.5) | 3.6-7.9 |
| OC, ng/ml | 18.6 (16.8, 21.1) | 28.9 (26.0, 35.6) | 13.9-30.6 |
| CXCL13, pg/ml | 116.4 (98.2, 130.6) | 236.8 (180.8, 323.9) | 37.8-153.6 |
| sICAM-1, ng/ml | 257.7 (239.7, 272.3) | 339.4 (313.7, 380.0) | 186.0-331.0 |
| Iron μmol/l | 10.9 (9.8, 12.2) | 17.2 (15.5, 20.3) | 10.8-28.9 |
| Ferritin, ng/ml | 76.7 (60.5, 93.5) | 204.3 (154.9, 283.4) | 18.6-148.3 |
| TIBC, μg/dl | 322.0 (313.0, 332.0) | 373.0 (357.0, 397.0) | 247.2-363.0 |
| Hepcidin, ng/ml | 23.0 (17.0, 28.9) | 62.4 (43.9, 77.0) | 0.6-46.4 |

Data presented as median (Q1, Q3) at baseline. Reference range for CRP is based on population of healthy men and women (reference range provided by Covance); for all other biomarkers, reference range is based on healthy post-menopausal women (5th-95th percentile; reference ranges provided by Bioclinica).
*Unadjusted Wilcoxon test P-value <5%. CRP: C-reactive protein; CXCL13: chemokine (C-X-C motif) ligand 13; Lp(a): lipoprotein (a); MMP 3: matrix metalloproteinase-3; OC: osteocalcin; OPG: osteoprotegerin; P1NP: procollagen type 1 N-terminal propeptide; Q: quartile; q2w: every 2 weeks; RANKL: receptor activator of nuclear factor-κB ligand; SAA: serum amyloid A; sICAM-1: soluble intercellular adhesion molecule-1; TIBC: total iron-binding capacity.

Biomarkers were analyzed retrospectively (except CRP) at one or two post-baseline timepoints through week 24 (Table 2). Timepoints selected for analysis were based on either previous data following sarilumab treatment or on literature suggesting either acute or latent effects of RA therapy on specific markers. The assay characteristics for most biomarkers have been described previously (Gabay C, 2018).

TABLE 2
Individual serum biomarker assessment schedule

| Function | Biomarker | Baseline | Week 2 | Week 4 | Week 8 | Week 12 | Week 16 | Week 20 | Week 24 |
|---|---|---|---|---|---|---|---|---|---|
| Acute-phase response | SAA | X | | | | X | | | X |
| | CRP | X | | X | X | X | X | X | X |
| Atherothrombosis | Lp(a) | X | | | | X | | | X |
| Synovial inflammation | MMP-3 | X | | | | | | | X |
| Bone remodeling | Total RANKL | X | X | | | | | | X |
| | P1NP | X | | | | | | | X |
| | OPG | X | X | | | | | | X |
| | Osteocalcin | X | | | | | | | X |
| Marker reflecting synovial | CXCL13 | X | X | | | | | | X |
| lymphoid cell infiltrate | | | | | | | | | |
| Marker reflecting synovial | sICAM-1 | X | X | | | | | | X |
| myeloid cell infiltrate | | | | | | | | | |
| Anemia of chronic disease | Iron | X | X | | | | | | |
| | Ferritin | X | X | | | | | | |
| | TIBC | X | X | | | | | | |
| | Hepcidin | X | X | | | | | | |

CRP: C-reactive protein; CXCL13: chemokine (C-X-C motif) ligand 13; Lp(a): lipoprotein (a); MMP-3: matrix metalloproteinase-3; OPG: osteoprotegerin; P1NP: procollagen type 1 N-terminal propeptide; RANKL: receptor activator of nuclear factor-κB ligand; SAA: serum amyloid A; sICAM-1: soluble intercellular adhesion molecule-1; TIBC: total iron-binding capacity

### Statistical Analysis

Baseline biomarker levels were compared between treatment groups using a Wilcoxon test. Spearman's ranked correlations at baseline were computed in the overall biomarker population. To evaluate pharmacodynamic changes in circulating biomarker concentrations between treatment groups at each timepoint, absolute and percentage changes from baseline were described. In addition, the percentage changes in biomarker concentrations were analyzed using non-parametric methods because of non-normal distributions. For biomarkers measured once postbaseline, a rank-based analysis of covariance (ANCOVA) adjusted on baseline value was implemented. For biomarkers measured twice post-baseline, a mixed-effect model with repeated measures was performed on rank-transformed data (analysis of variance [ANOVA]-type method), with treatment, visit and treatment-by-visit interaction as fixed effects, baseline biomarker value transformed in rank, baseline biomarker value transformed in rank-by-visit interaction as fixed covariates, assuming an unstructured covariance structure. P-values were adjusted for false discovery rate (Benjamini-Hochberg 5% threshold). The number of patients with abnormal biomarker levels at baseline (according to the reference ranges provided by the testing laboratory) that normalized with treatment was compared between groups using a χ2 test; unadjusted P-values are reported.

Percentage changes in biomarker concentrations at week 24 were compared between clinical responders and non-responders at the same visit within each treatment group using similar non-parametric methods. P-values were also adjusted for false discovery rate.

For binary efficacy endpoints, predictive effects of baseline biomarker values on sarilumab efficacy vs adalimumab were tested using a logistic regression with treatment group and region as fixed effects, baseline biomarker value as a continuous covariate, and the baseline biomarker-by-treatment group interaction. For continuous PROs, a linear regression was used with the same effects as above, as well as the baseline PRO value as a covariate. Unadjusted P-values for the interaction are reported to assess the predictive value of the biomarkers. Similar analyses were performed after categorization of patients into high, medium and low biomarker levels at baseline using tertile values in the biomarker population. In addition, pairwise comparisons of responses between sarilumab and adalimumab were performed separately in patients with high, medium and low biomarker levels, and the Mantel-Haenszel estimates of odds ratios (ORs), stratified by region, and 95% confidence intervals (CIs) were derived and graphically represented using forest plots. For continuous PROs, a linear regression was performed separately in each biomarker tertile and differences in least squares mean (LSM) changes with 95% CI between both treatments were provided.

Differential combinations of circulating CXCL13 and sICAM-1 (low or high levels defined relative to baseline median levels) were assessed for prediction of response to sarilumab, using Mantel-Haenszel estimates of ORs derived for each combination.

All analyses were performed using SAS version 9.2 or higher (SAS Institute Inc., Cary, NC, USA).

### Biomarker Analysis

All biomarker serum concentrations, except C-reactive protein (CRP), were analyzed retrospectively using a validated proprietary enzyme-linked immunosorbent assay (ELISA) at Bioclinica Lab (Lyon, France). CRP was assessed at Covance Laboratories (Indianapolis, IN, USA, Geneva, Switzerland or Singapore) using the Siemens high-sensitivity CRP nephelometry assay. The intra-assay precision was <3%, inter-assay precision was <5.4%, and the reference range for healthy controls was ≤2.87 mg/l. Serum levels of chemokine (C—X—C motif) ligand 13 and soluble intercellular adhesion molecule-1 were assessed using an ELISA (Quantikine® ELISA kit, R&D Systems, Minneapolis, MN, USA), with inter-assay coefficient of variation (CV)<8% and intra-assay CV<15%. Serum procollagen type 1 N-terminal propeptide (PINP) was measured using the Roche Modular S PINP assay, with intra- and inter-assay CVs<7%. Serum amyloid A was measured using an ELISA (Anogen) with intra- and inter-assay CVs<7%. Ferritin was measured using the Roche Modular Serum Ferritin assay, with intra- and inter-assay CVs<3%. Total iron-binding capacity was measured using a Kone 20 analyser, Konelab (Total Iron-Binding Capacity [RANDOX]) with intra- and inter-assay CVs<5.5% and <4.7%, respectively. Serum levels of iron were measured using a Kone 20 analyzer, Konelab (Iron [Thermo Scientific]) with intra- and inter-assay CVs<7.8% and <6%, respectively. Hepcidin levels were measured using an ELISA (Human Hepcidin 25 (bioactive) HS ELISA [DRG]) with intra- and inter-assay CVs<9.6% and <8.1%, respectively. Biomarker levels below the lower limit of quantification (LLOQ) were replaced by LLOQ/2 in all analyses, and those above upper limit of quantification (ULOQ) by ULOQ.

### Baseline Demographics, Disease Characteristics, Efficacy and Biomarker Levels

Baseline demographics and disease characteristics of the biomarker population were generally similar to the overall intent-to-treat (ITT) population (Table 2). Overall, efficacy and PROs were also generally similar between the ITT and biomarker populations (Table 3).

TABLE 3
Efficacy and PROs at week 24 in the biomarker and
ITT populations

| ITT population | | Biomarker population | |
|---|---|---|---|
| Adalimumab | Sarilumab | Adalimumab | Sarilumab |
| 40 mg | 200 mg | 40 mg | 200 mg |
| q2w | q2w | q2w | q2w |
| (n = 185) | (n = 184) | (n = 154) | (n = 153) |

Efficacy results at week 24^{a}

| | | | | |
|---|---|---|---|---|
| ACR20 | 58.4 | 71.7 | 59.1 | 73.9 |
| responders, | | | | |
| % | | | | |
| ACR50 | 29.7 | 45.7 | 29.2 | 49.7 |
| responders, | | | | |
| % | | | | |
| ACR70 | 11.9 | 23.4 | 12.3 | 25.5 |
| responders, | | | | |
| % | | | | |
| ΔDAS28- | −2.2 | −3.4 | −2.3 | −3.4 |
| ESR | (1.4) | (1.4) | (1.3) | (1.3) |
| DAS28- | 7.0 | 26.6 | 8.4 | 27.5 |
| ESR < | | | | |
| 2.6, % | | | | |
| DAS28- | 14.1 | 42.9 | 15.6 | 43.8 |
| ESR < | | | | |
| 3.2, % | | | | |
| ΔDAS28- | −2.1 | −2.9 | −2.1 | −3.0 |
| CRP | (1.2) | (1.3) | (1.3) | (1.2) |
| DAS28- | 13.5 | 34.2 | 13.0 | 34.6 |
| CRP < | | | | |
| 2.6, % | | | | |
| DAS28- | 24.3 | 51.6 | 24.0 | 52.3 |
| CRP < | | | | |
| 3.2, % | | | | |
| ΔTJC | −16.4 | −19.0 | −16.6 | −19.4 |
| | (12.0) | (13.3) | (12.3) | (12.5) |
| ΔSJC | −12.2 | −14.3 | −12.1 | −14.6 |
| | (9.1) | (9.6) | (8.9) | (9.8) |
| ΔCDAI | −25.5 | −29.7 | −25.9 | −30.2 |
| | (12.9) | (12.7) | (13.3) | (12.0) |
| CDAI ≤ | 2.7 | 7.1 | 3.2 | 7.2 |
| 2.8, % | | | | |
| CDAI ≤ | 24.9 | 41.8 | 24.7 | 43.1 |
| 10, % | | | | |
| ΔPhysician | −37.3 | −45.3 | −37.6 | −45.7 |
| global | (22.5) | (21.4) | (22.8) | (20.5) |
| VAS | | | | |
| (0-100 mm) | | | | |

PROs at week 24^{a}

| | | | | |
|---|---|---|---|---|
| ΔHAQ-DI | −0.4 | −0.6 | −0.4 | −0.7 |
| | (0.6) | (0.7) | (0.6) | (0.7) |
| ΔFACIT- | 8.2 | 10.4 | 8.4 | 11.3 |
| Fatigue | (10.4) | (10.2) | (10.3) | (10.0) |
| (0-52) | | | | |
| ΔPatient | −25.0 | −33.5 | −25.5 | −33.8 |
| global | (25.2) | (26.2) | (24.9) | (26.3) |
| VAS | | | | |
| (0-100 mm) | | | | |
| ΔPain VAS | −27.9 | −36.4 | −28.0 | −36.9 |
| (0-100 mm) | (24.7) | (26.9) | (24.4) | (27.0) |
| ΔSF-36 | 5.5 | 8.6 | 5.3 | 9.0 |
| PCS | (7.1) | (7.7) | (7.1) | (7.6) |
| ΔSF-36 | 7.0 | 8.2 | 6.9 | 8.5 |
| MCS | (11.3) | (10.8) | (11.0) | (11.3) |
| ΔMorning | −27.0 | −36.1 | −26.7 | −36.7 |
| stiffness | (27.4) | (27.9) | (27.8) | (27.3) |
| VAS | | | | |
| (0-100 mm) | | | | |
| ΔRAID | −2.1 | −3.2 | −2.1 | −3.3 |
| (0-10) | (2.4) | (2.4) | (2.4) | (2.4) |

Mean (standard deviation) unless otherwise stated.
Δ: absolute change from baseline;
ACR20/50/70: American College of Rheumatology 20/50/70% improvement criteria;
CDAI: Clinical Disease Activity Index;
DAS28-CRP: Disease Activity Score (28 joints) using C-reactive protein;
DAS28-ESR: Disease Activity Score (28 joints) using erythrocyte sedimentation rate;
FACIT: Functional Assessment of Chronic Illness Therapy;
HAQ-DI: Health Assessment Questionnaire-Disability Index;
ITT: intent-to-treat;
MCS: mental component summary;
PCS: physical component summary;
PRO: patient-reported outcome;
q2w: every 2 weeks;
RAID: rheumatoid arthritis impact of disease;
SJC: swollen joint count;
TJC: tender joint count;
VAS: visual analogue scale.

Baseline serum levels of most biomarkers were similar between treatment groups, except for Lp(a), which was higher in the adalimumab than the sarilumab groups (Lp[a]: median 235.5 vs 179.0 mg/l, respectively; Wilcoxon test P-value: 0.039; Table 1).

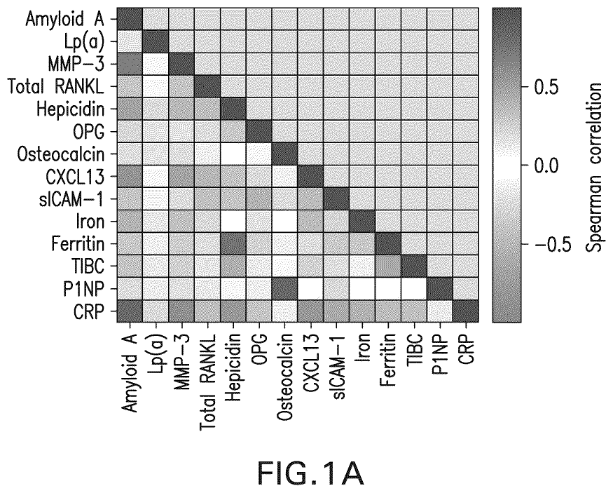

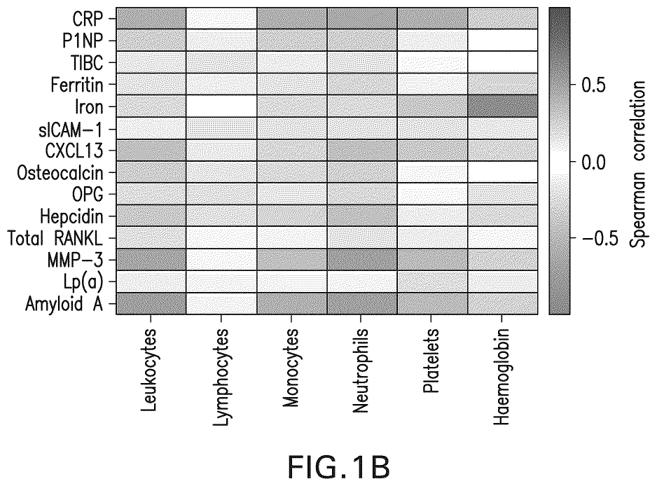

Correlations between individual biomarkers at baseline were generally low or moderate (ρ<0.5; FIG. 1A). Correlation coefficients above 0.7 were observed for markers of inflammation (CRP and SAA; ρ=0.81), bone formation (PINP and OC; ρ=0.82), and anemia of chronic disease (ferritin and hepcidin; ρ=0.74), as expected. Moderate correlations were observed between baseline CRP, SAA or MMP-3 with differential blood counts (leucocytes and neutrophils; ρ from 0.4 to 0.5), and, as expected, between iron and haemoglobin (ρ=0.57; FIG. 1B).

### Pharmacodynamic Effects of Treatment on Biomarkers

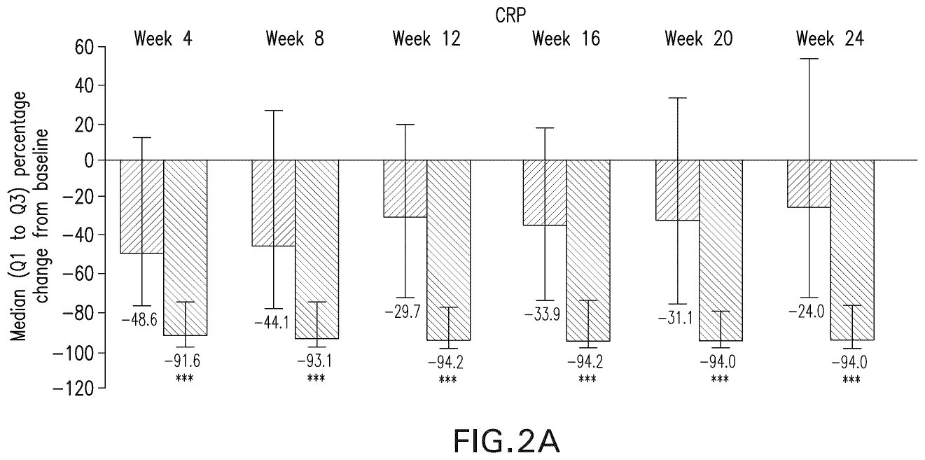

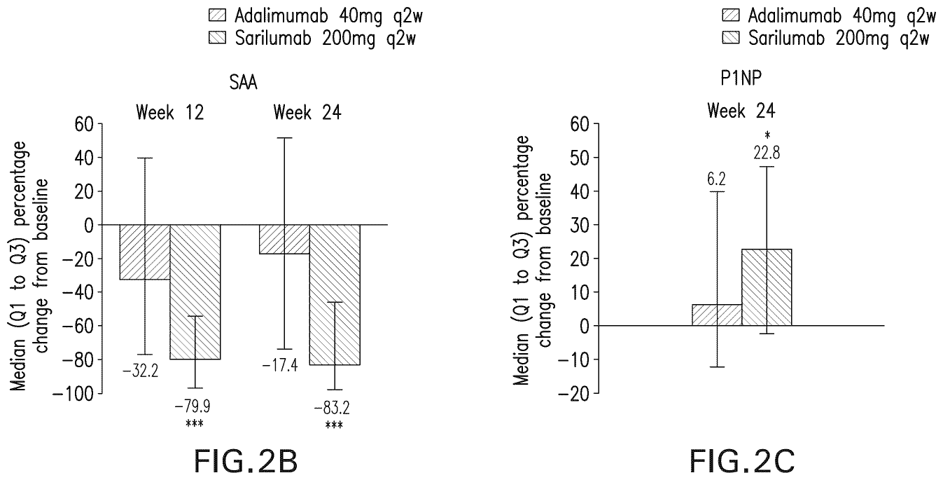

To compare the effects of sarilumab and adalimumab treatment on biomarkers over time, the absolute (Table 4) and percentage changes from baseline in biomarker concentrations were analyzed up to week 24. Greater reductions in biomarkers associated with the acute-phase response were observed at weeks 12 and 24 following treatment with sarilumab vs adalimumab (adjusted P<0.0001; FIGS. 2A and 2B). Reductions in CRP were observed as early as week 4 with sarilumab vs adalimumab and were sustained throughout the treatment period (FIG. 2A).

TABLE 4
Absolute change from baseline in biomarker concentrations through week 24

| Median absolute change | | | | |
|---|---|---|---|---|
| from baseline (Q1, Q3) | | Week 2 | Week 12 | Week 24 |
| SAA, ng/ml | Adalimumab | — | −2442.7 | −476.4 |
| | 40 mg q2w | | (−22330.7, 1930.9) | (−19654.3, 3342.1) |
| | Sarilumab | — | −9066.5 | −9604.7 |
| | 200 mg q2w | | (−80132.7, −1902.8) | (−79255.8, −1398.0) |
| CRP, mg/l | Adalimumab | — | −1.3 | −1.3 |
| | 40 mg q2w | | (−13.7, 1.3) | (−13.7, 3.5) |
| | Sarilumab | — | −6.8 | −6.6 |
| | 200 mg q2w | | (−22.9, −1.6) | (−22.8, −1.3) |
| Lp(a), mg/l | Adalimumab | — | −2.0 | −4.5 |
| | 40 mg q2w | | (−54.0, 28.0) | (−40.0, 28.0) |
| | Sarilumab | — | −59.0 | −60.3 |
| | 200 mg q2w | | (−134.0, −13.0) | (−157.0, −21.0) |
| MMP-3, | Adalimumab | — | — | −5.6 |
| ng/ml | 40 mg q2w | | | (−23.6, 6.3) |
| | Sarilumab | — | — | −6.8 |
| | 200 mg q2w | | | (−33.1, 1.4) |
| Total | Adalimumab | 15.9 | — | 31.5 |
| RANKL, | 40 mg q2w | (−7.2, 129.1) | | (−93.5, 223.2) |
| pmol/l | Sarilumab | −10.5 | — | −76.8 |
| | 200 mg q2w | (−69.5, 31.0) | | (−438.5, 20.4) |
| P1NP, | Adalimumab | — | — | 2.0 |
| ng/ml | 40 mg q2w | | | (−5.5, 13.5) |
| | Sarilumab | — | — | 8.6 |
| | 200 mg q2w | | | (−0.8, 18.7) |
| OPG, | Adalimumab | −0.2 | — | 0.2 |
| pmol/l | 40 mg q2w | (−0.7, 0.3) | | (−0.6, 0.9) |
| | Sarilumab | 0.1 | — | 0.1 |
| | 200 mg q2w | (−0.5, 0.7) | | (−0.7, 0.7) |
| OC, | Adalimumab | — | — | 0.9 |
| ng/ml | 40 mg q2w | | | (−1.7, 5.1) |
| | Sarilumab | — | — | 2.4 |
| | 200 mg q2w | | | (−1.4, 5.7) |
| CXCL13, | Adalimumab | −45.4 | — | −30.5 |
| pg/ml | 40 mg q2w | (−81.7, −22.9) | | (−65.7, −1.5) |
| | Sarilumab | −12.8 | — | −35.7 |
| | 200 mg q2w | (−41.0, 5.6) | | (−80.5, −4.5) |
| sICAM-1 | Adalimumab | −23.2 | — | −10.8 |
| | 40 mg q2w | (−37.8, −9.3) | | (−41.7, 14.4) |
| | Sarilumab | −0.7 | — | −11.0 |
| | 200 mg q2w | (−15.6, −12.8) | | (−36.7, 1.9) |
| Iron, | Adalimumab | 1.4 | — | — |
| μmol/l | 40 mg q2w | (−0.6, 4.5) | | |
| | Sarilumab | 3.7 | — | — |
| | 200 mg q2w | (0.5, 8.3) | | |
| Ferritin, | Adalimumab | −8.5 | — | — |
| ng/ml | 40 mg q2w | (−31.7, 0.5) | | |
| | Sarilumab | −7.7 | — | — |
| | 200 mg q2w | (−26.4, 0.1) | | |
| TIBC, | Adalimumab | 9.0 | — | — |
| μg/dl | 40 mg q2w | (−2.0, 22.0) | | |
| | Sarilumab | 20.0 | — | — |
| | 200 mg q2w | (5.0, 35.0) | | |
| Hepcidin, | Adalimumab | −5.8 | — | — |
| ng/ml | 40 mg q2w | (−19.1, −0.1) | | |
| | Sarilumab | −3.3 | — | — |
| | 200 mg q2w | (−16.3, 0.4) | | |

Sample size in the overall biomarker population: adalimumab 40 mg q2w: n = 154, sarilumab 200 mg q2w: n = 153.
CRP: C-reactive protein;
CXCL13: chemokine (C-X-C motif) ligand 13;
Lp(a): lipoprotein (a);
MMP 3: matrix metalloproteinase-3;
OC: osteocalcin;
OPG: osteoprotegerin;
P1NP: procollagen type 1 N-terminal propeptide;
Q: quartile;
q2w: every 2 weeks;
RANKL: receptor activator of nuclear factor-κB ligand;
SAA: serum amyloid A;
sICAM-1: soluble intercellular adhesion molecule-1;
TIBC: total iron-binding capacity

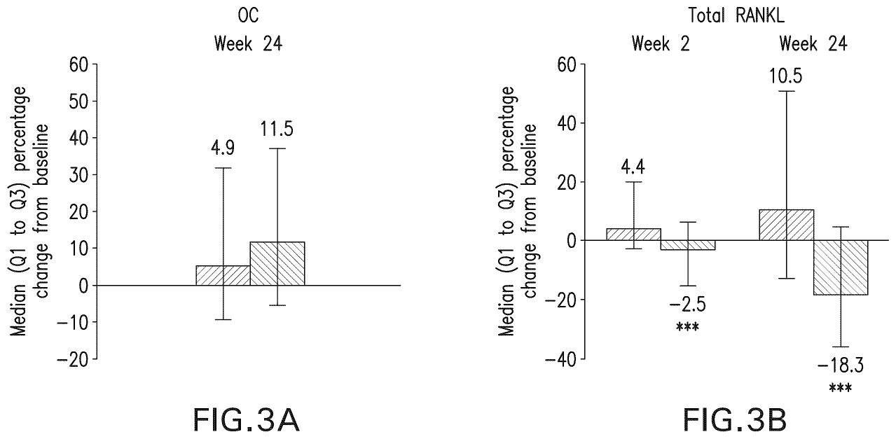

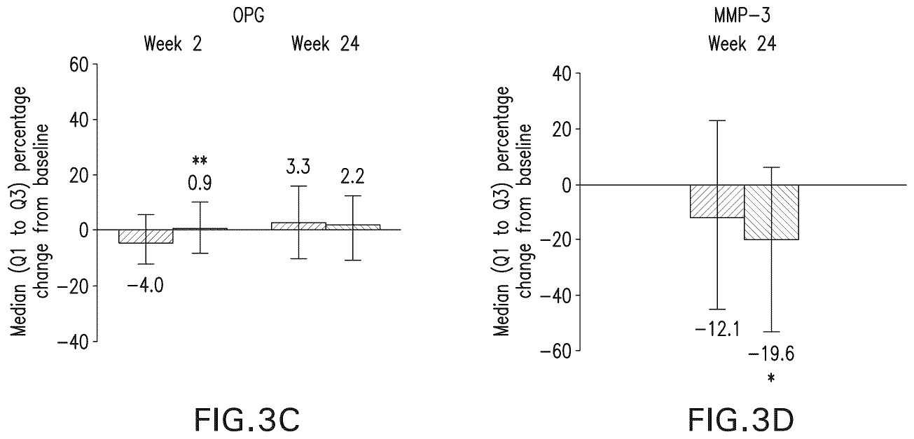

At week 24, sarilumab treatment increased concentrations of PINP, a marker of osteoblast activation, compared with adalimumab (adjusted P=0.027; FIG. 2C). A numeric increase in OC, another marker of osteoblast activity, was also observed in sarilumab-vs adalimumab-treated patients (FIG. 3A). Furthermore, reductions in total RANKL, a marker of bone resorption, were observed as early as week 2 with sarilumab compared with adalimumab and persisted through week 24 (adjusted P<0.0001; FIG. 3B); in addition, a numeric increase in total RANKL was observed after adalimumab treatment. A transient decrease in OPG, a decoy for RANKL, was observed after adalimumab treatment at week 2 but did not persist through week 24 (FIG. 3C). Additionally, greater reductions in MMP-3 were observed with sarilumab at week 24 (adjusted P=0.020; FIG. 3D).

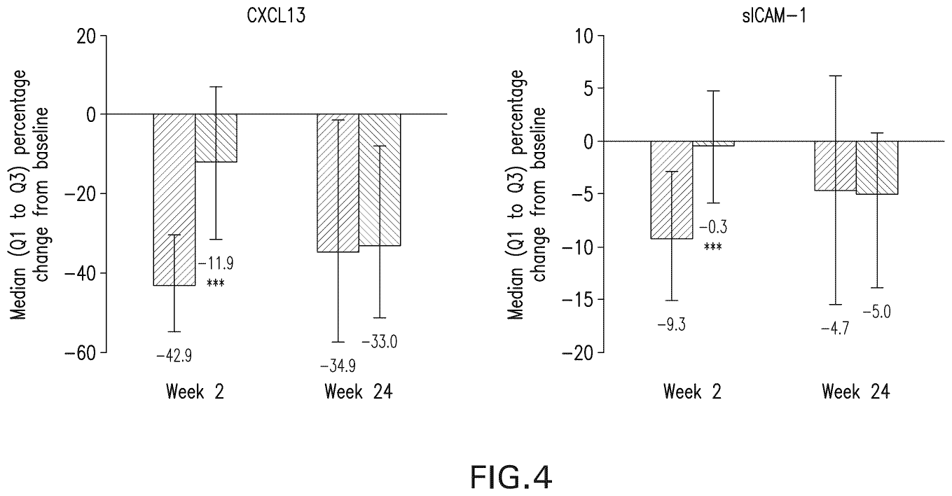

The effects of sarilumab and adalimumab on biomarkers associated with markers purported to reflect synovial lymphoid and myeloid cell infiltrates, CXCL13 and sICAM-1, respectively, were also examined. While greater reductions in these biomarkers were observed 2 weeks post-treatment with adalimumab vs sarilumab, these effects did not persist through week 24 (FIG. 4).

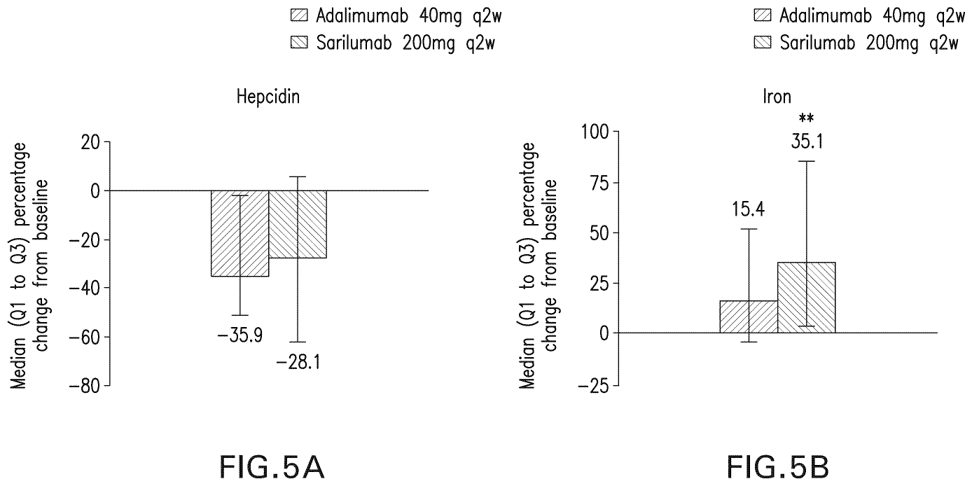

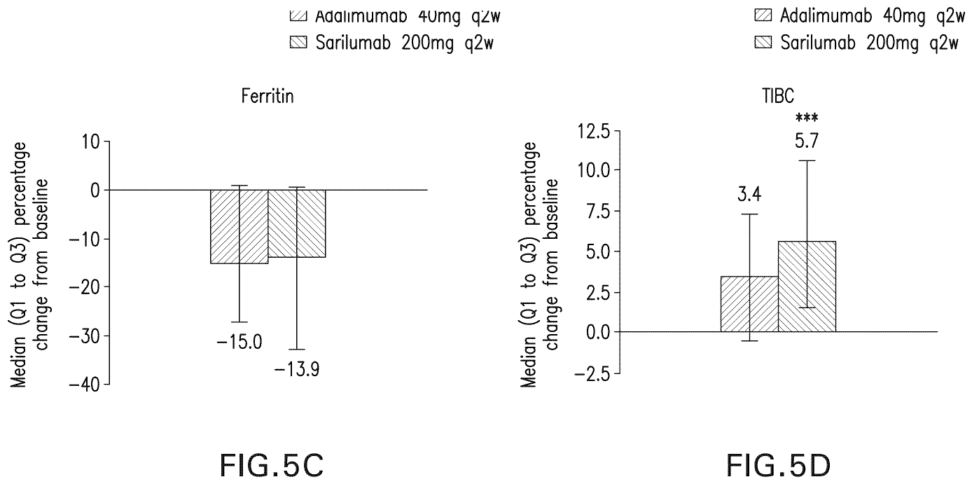

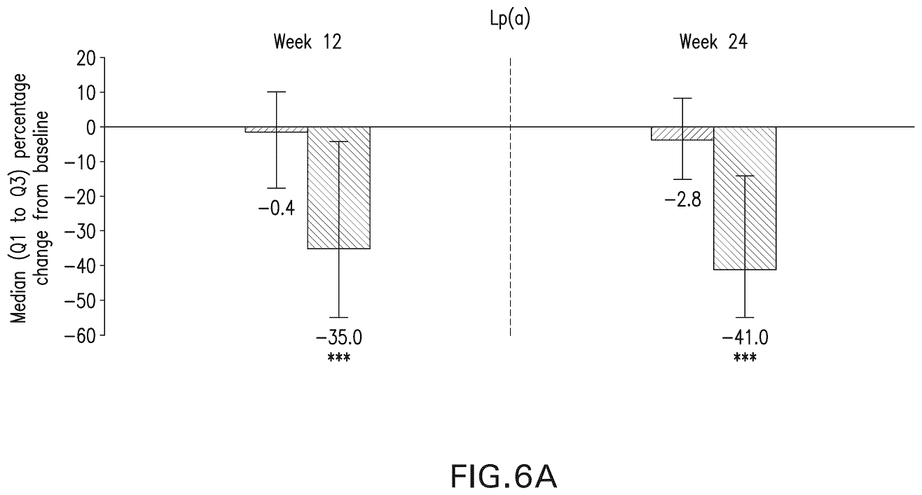

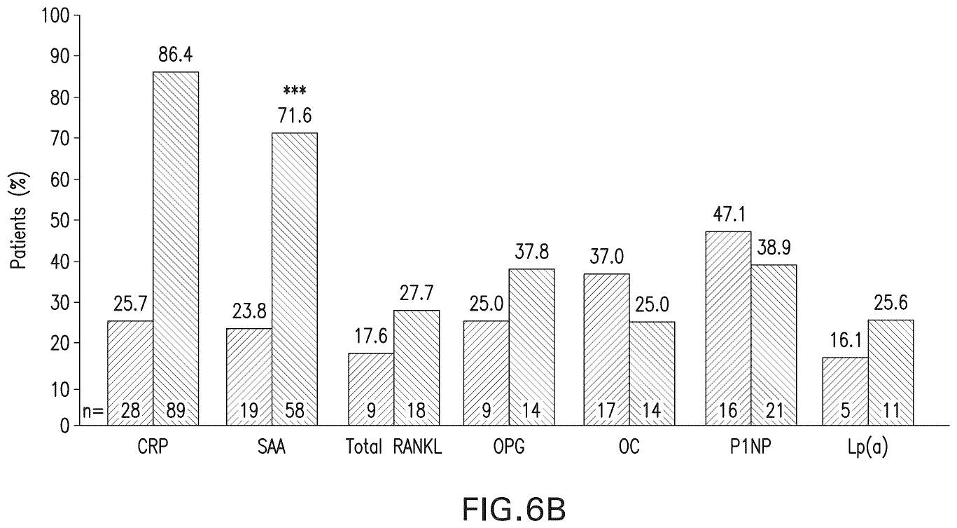

We also examined the effects of treatment on parameters associated with anemia of chronic disease. In the ITT population (Burmester G R, Arthritis Rheumatol 2018; 70), sarilumab resulted in larger increases in haemoglobin levels vs adalimumab at week 24 (LSM changes from baseline 0.59 vs 0.08 g/dl; LSM difference 0.52 g/dl [95% CI: 0.32, 0.71; nominal P<0.001]). In this analysis, reductions in hepcidin and ferritin were observed at week 2 with both sarilumab and adalimumab. In contrast, increases in iron and TIBC were observed with sarilumab relative to adalimumab at week 2 post-treatment (FIGS. 5A-5D). Reductions in the lipid particle Lp(a) were observed with sarilumab vs adalimumab at week 24 (adjusted P<0.0001; FIG. 6A).

A subset of patients had abnormal baseline biomarker levels relative to reference ranges. In these patients, normalization of CRP and SAA was evident in a greater percentage treated with sarilumab than adalimumab at week 24 (unadjusted P<0.0001). Normalization of total RANKL, OPG and Lp(a) occurred in a numerically greater percentage of patients treated with sarilumab vs adalimumab at week 24 (FIG. 6B).

### Relationship Between Changes in Biomarker Levels and Clinical Responses

To establish whether post-treatment changes in biomarker levels at week 24 were associated with clinical efficacy, changes were compared between sarilumab- and adalimumab-treated responders and non-responders. Median percentage changes at week 24 in total RANKL, OPG, PINP, OC and Lp(a) did not differ greatly between responders and non-responders (data not shown). However, reductions in SAA from baseline at week 24 were greater in adalimumab ACR20 and DAS28-CRP<3.2 responders than non-responders (−33.3% vs 0.0%, respectively; nominal P=0.0038 and −39.2% vs 0.0%, respectively; nominal P=0.0061, respectively). Greater reductions in MMP-3 were also observed in adalimumab ACR20 responders vs non-responders (−23.6% vs 17.1%, respectively; nominal P<0.0001). Associations between clinical efficacy and changes from baseline in SAA and MMP-3 were not observed in sarilumab-treated patients, and although both responders and non-responders had a ≥90% reduction in CRP, the P-values for comparisons of responders vs non-responders were <0.05 across several parameters, including ACR20/50, DAS28-CRP<3.2 and DAS28-CRP<2.6 (data not shown).

### Correlations Between Biomarkers and Disease Activity and PROs at Baseline

The strongest correlations between baseline biomarkers and baseline disease activity were observed for SAA and CRP with DAS28-ESR (ρ=0.26 and 0.31, respectively) and for CRP, SAA, MMP-3, hepcidin and CXCL13 with DAS28-CRP (p from 0.36 to 0.58). None of the biomarkers correlated with baseline PROs (all ρ<0.3).

### Predictive Analysis of Baseline Biomarker Levels on Clinical Responses and PROs

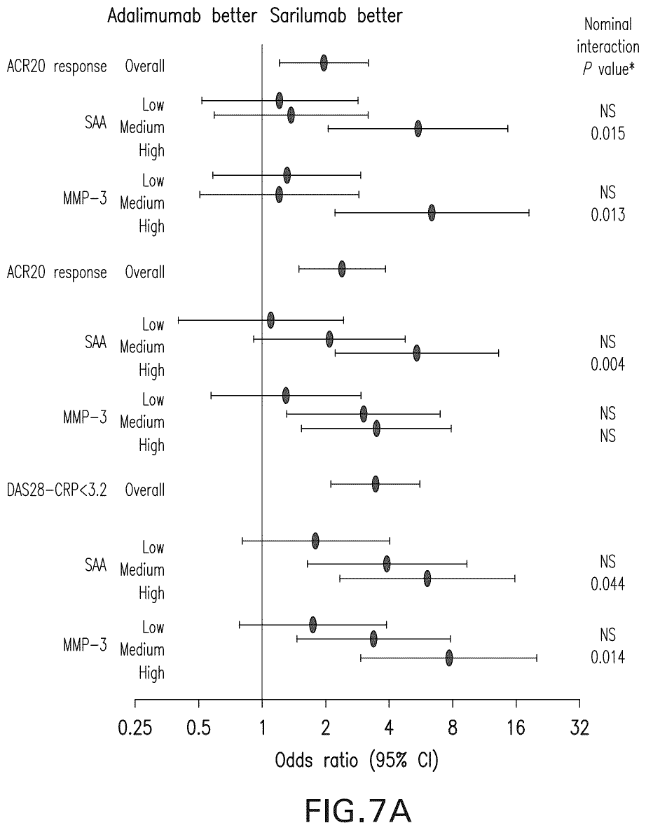

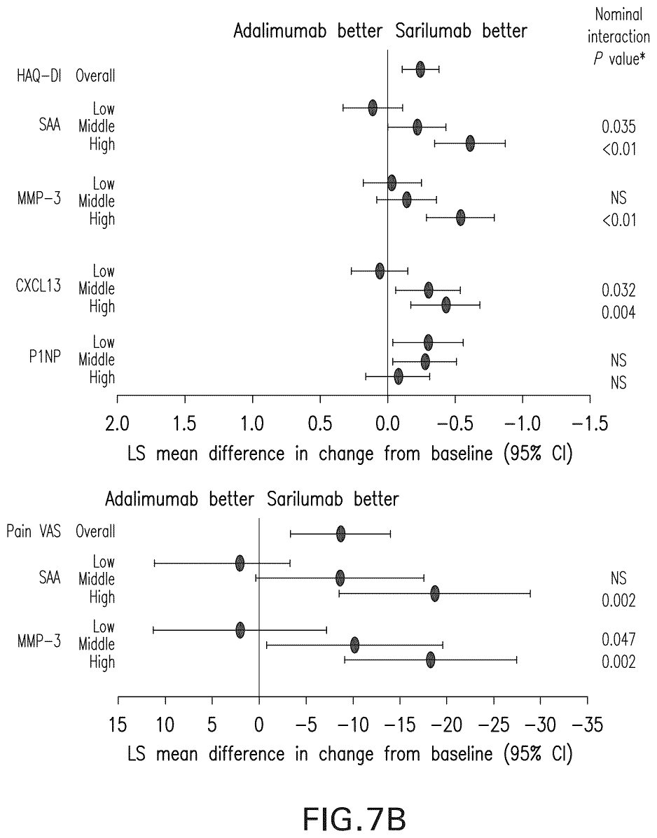

Baseline biomarker levels were analyzed as continuous and categorical measures by tertiles (low, medium and high) because thresholds associated with clinical efficacy are not currently established, and treatment-by-biomarker interaction P-values were calculated to assess the predictivity of the biomarker. Treatment-by-tertile biomarker interactions for efficacy endpoints at week 24 analyzed by baseline biomarkers in tertiles are shown in FIG. 7A and Table 5. Patients with the highest baseline concentrations of SAA who received sarilumab were more likely to achieve ACR20/50/70 or DAS28-CRP<3.2 responses than with adalimumab compared with patients in the low tertile: ACR20 (OR [95% CI] 5.5 [2.1, 14.5]); ACR50 (5.4 [2.2, 13.2]); ACR70 (5.7 [1.8, 18.4]); DAS28-CRP<3.2 (6.1 [2.3, 15.7]) (FIG. 7A and Table 5). SAA was consistently predictive compared with high MMP-3 and CRP, which were only predictive of ACR20 and DAS28-CRP<3.2 response (Table 5). Baseline levels of biomarkers associated with bone remodeling, synovial lymphoid and myeloid cell infiltrates and anemia of inflammation were not predictive of efficacy at week 24, except for hepcidin and CXCL13, which were associated with ACR20 response.

TABLE 5
Treatment-by-tertile biomarker interactions for efficacy endpoints
at week 24 analyzed by baseline biomarkers in tertiles

| Efficacy | Biomarker at baseline | | | | | | | | | | | | | |
|---|---|---|---|---|---|---|---|---|---|---|---|---|---|---|
| endpoint at | SAA | | CRP | | MMP-3 | | OPG | | OC | | CXCL13 | | Hepcidin | |
| week 24 | M/L | H/L | M/L | H/L | M/L | H/L | M/L | H/L | M/L | H/L | M/L | H/L | M/L | H/L |
| ACR20 | NS | 0.015 | NS | 0.039 | NS | 0.013 | NS | NS | 0.031 | NS | NS | 0.003 | NS | 0.021 |
| ACR50 | NS | 0.004 | NS | NS | NS | NS | NS | NS | NS | NS | NS | NS | NS | NS |
| ACR70 | NS | 0.008 | NS | NS | NS | NS | 0.032 | NS | NS | NS | NS | NS | NS | NS |
| DAS28-ESR <2.6 | NS | NS | NS | NS | NS | NS | NS | NS | NS | NS | NS | NS | NS | NS |
| DAS28-ESR <3.2 | 0.004 | NS | NS | NS | NS | NS | NS | NS | NS | NS | NS | NS | NS | NS |
| DAS28-CRP <2.6 | NS | 0.041 | NS | NS | NS | NS | NS | NS | NS | NS | NS | NS | NS | NS |
| DAS28-CRP <3.2 | NS | 0.044 | NS | 0.049 | NS | 0.014 | NS | NS | NS | NS | NS | NS | NS | NS |

M/L: Nominal treatment-by-biomarker interaction P-value for Medium vs Low tertile; H/L: Nominal treatment-by-biomarker interaction P-value for High vs Low tertile. ACR20/50/70: American College of Rheumatology 20/50/70% responses; CRP: C-reactive protein; CXCL13: chemokine (C-X-C motif) ligand 13; DAS28 CRP: Disease Activity Score (28 joints) C-reactive protein; DAS28-ESR: Disease Activity Score (28 joints) erythrocyte sedimentation rate; MMP-3: matrix metalloproteinase 3; NS: not significant at 5%; OC: osteocalcin; OPG: osteoprotegerin; SAA: serum amyloid A.

The ability of baseline biomarker levels to predict PRO responses was also analyzed by their respective tertiles and showed that sarilumab-treated patients with higher SAA, MMP-3 and hepcidin levels reported improved PRO responses including HAQ-DI (FIG. 7B top) and pain VAS (FIG. 7B bottom) scores compared with adalimumab-treated patients, as well as patient global VAS, morning stiffness VAS, SF-36 PCS and physical functioning domain and RAID. P-values for these interactions are included in FIG. 7B and Table 6 to demonstrate the differential efficacy predicted by high levels of these biomarkers compared with low levels. Baseline levels of markers associated with anemia of chronic disease (hepcidin, ferritin and iron) were also associated with PRO improvements at week 24 (Table 6). Analysis of biomarkers as continuous measures also revealed interactions for SAA, MMP-3, CRP and PINP with HAQ-DI at week 24 (interaction nominal P-values<0.01).

TABLE 6
Treatment-by-tertile biomarker interactions for PROs at week 24 analyzed by baseline biomarkers in tertiles

| Change from | | | | | | | | | | | | | | | | | | |
|---|---|---|---|---|---|---|---|---|---|---|---|---|---|---|---|---|---|---|
| baseline in | Biomarker at baseline | | | | | | | | | | | | | | | | | |
| PROs at | SAA | | CRP | | MMP-3 | | OC | | CXCL13 | | Hepcidin | | s-ICAM1 | | Iron | | Ferritin | |
| Week 24 | M/L | H/L | M/L | H/L | M/L | H/L | M/L | H/L | M/L | H/L | M/L | H/L | M/L | H/L | M/L | H/L | M/L | H/L |
| Patient | NS | 0.011 | 0.002 | 0.040 | NS | 0.010 | NS | NS | NS | NS | NS | 0.009 | NS | NS | NS | NS | NS | NS |
| global | | | | | | | | | | | | | | | | | | |
| VAS | | | | | | | | | | | | | | | | | | |
| HAQ-DI | 0.035 | <0.001 | NS | 0.005 | NS | <0.001 | NS | NS | 0.032 | 0.004 | NS | NS | NS | NS | NS | 0.032 | NS | NS |
| Pain VAS | NS | 0.002 | 0.021 | 0.029 | 0.047 | 0.002 | NS | NS | NS | NS | 0.010 | 0.002 | NS | NS | NS | NS | NS | NS |
| SF-36 - | NS | <0.001 | 0.009 | 0.016 | NS | 0.026 | NS | NS | NS | 0.031 | NS | NS | NS | NS | NS | NS | NS | NS |
| PCS score | | | | | | | | | | | | | | | | | | |
| SF-36 - | NS | NS | NS | NS | NS | NS | NS | NS | NS | NS | 0.030 | 0.023 | NS | NS | NS | NS | NS | 0.050 |
| MCS score | | | | | | | | | | | | | | | | | | |
| SF-36 - | NS | 0.003 | NS | NS | NS | 0.036 | NS | NS | NS | 0.003 | NS | NS | NS | NS | NS | NS | NS | NS |
| PF domain | | | | | | | | | | | | | | | | | | |
| SF-36 - | NS | NS | NS | NS | NS | NS | 0.037 | NS | NS | NS | 0.002 | 0.016 | NS | NS | NS | NS | NS | NS |
| BP domain | | | | | | | | | | | | | | | | | | |
| SF-36 - | NS | NS | NS | NS | NS | NS | NS | NS | NS | NS | 0.043 | 0.005 | NS | NS | NS | NS | NS | NS |
| VT domain | | | | | | | | | | | | | | | | | | |
| SF-36 - | NS | NS | 0.049 | NS | NS | NS | NS | NS | NS | NS | NS | NS | NS | 0.030 | NS | NS | NS | NS |
| RE domain | | | | | | | | | | | | | | | | | | |
| SF-36 - | NS | NS | NS | NS | NS | NS | NS | NS | NS | NS | 0.004 | 0.004 | NS | NS | NS | NS | 0.047 | NS |
| MH domain | | | | | | | | | | | | | | | | | | |
| Morning | NS | 0.004 | 0.002 | 0.017 | NS | <0.001 | NS | NS | NS | NS | 0.029 | <0.001 | NS | NS | NS | NS | NS | NS |
| stiffness | | | | | | | | | | | | | | | | | | |
| VAS | | | | | | | | | | | | | | | | | | |
| RAID score | NS | 0.017 | 0.045 | NS | NS | 0.020 | NS | NS | NS | NS | NS | 0.009 | 0.032 | NS | NS | NS | NS | NS |

M/L: Nominal treatment-by-biomarker interaction P-value for Medium vs Low tertile; H/L: Nominal treatment-by-biomarker interaction P-value for High vs Low tertile. BP: bodily pain; CRP: C-reactive protein; CXCL13: chemokine (C-X-C motif) ligand 13; FACIT: Functional Assessment of Chronic Illness Therapy; HAQ-DI: Health Assessment Questionnaire-Disability Index; Lp(a): lipoprotein (a); MCS: mental component summary; MH: mental health; MMP-3: matrix metalloproteinase-3; NS: not significant at 5%; OC: osteocalcin; OPG: osteoprotegerin; P1NP: procollagen type 1 N-terminal propeptide; PCS: physical component summary; PF: physical functioning; RAID: rheumatoid arthritis impact of disease; RANKL: receptor activator of nuclear factor-κB ligand; RE: role-emotional; SAA: serum amyloid A; SF-36: Medical Outcomes Study Short-Form (36-item) Health Survey; sICAM-1: soluble intercellular adhesion molecule-1; TIBC: total iron-binding capacity; VAS: visual analogue scale; VT: vitality.

Evaluation of Differential Combinations of Markers Associated with Myeloid and Lymphoid Activation for Prediction of Clinical Response

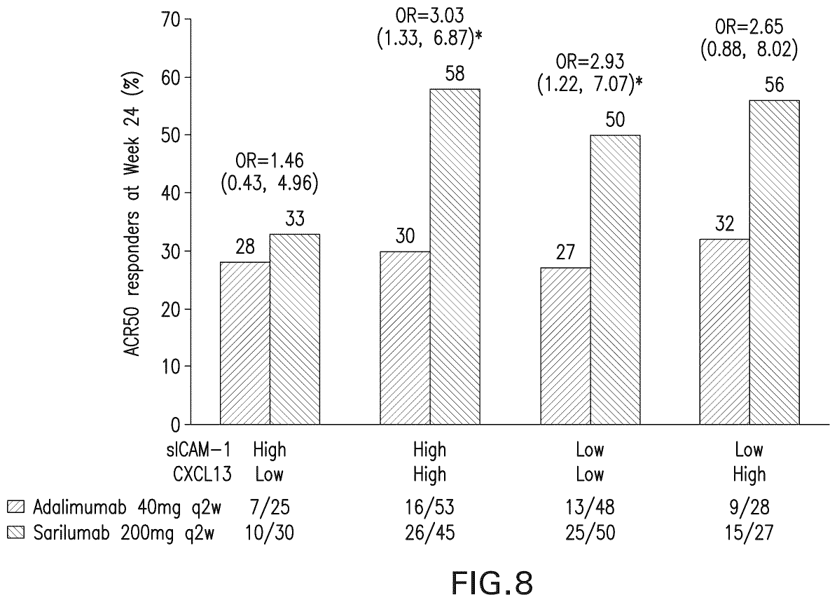

Baseline levels of CXCL13 and sICAM-1 were analyzed to determine whether differential ratios of these biomarkers (high/high, high/low, low/high and low/low; using the median in the overall population as the cut-off) could predict clinical responses to sarilumab or adalimumab treatment at week 24. While CXCL13 high/sICAM-1 high and CXCL13 low/sICAM-1 low patients had greater ACR50 responses with sarilumab than adalimumab, the other combinations were not predictive (nominal P>0.05; FIG. 8). Additionally, CXCL13 high/sICAM-1 high patients had greater ACR20 responses with sarilumab than adalimumab (OR 3.8 [95% CI 1.5, 9.8]; nominal P=0.004) but other combinations were not predictive (nominal P>0.05).

### REFERENCES

Karmakar S, Kay J, Gravallese E M. Bone damage in rheumatoid arthritis: mechanistic insights and approaches to prevention. Rheum Dis Clin North Am 2010; 36:385-404.
McInnes I B, Schett G. Cytokines in the pathogenesis of rheumatoid arthritis. Nat Rev Immunol 2007; 7:429-42.
Schett G, Gravallese E. Bone erosion in rheumatoid arthritis: mechanisms, diagnosis and treatment. Nature reviews. Rheumatology 2012; 8:656-64.
Choy E. Understanding the dynamics: pathways involved in the pathogenesis of rheumatoid arthritis. Rheumatology (Oxford) 2012; 51 Suppl 5: v3-11.
Smolen J S, Aletaha D. The assessment of disease activity in rheumatoid arthritis. Clin Exp Rheumatol 2010; 28 Suppl 59: S18-27.
Schett G. Physiological effects of modulating the interleukin-6 axis. Rheumatology (Oxford) 2018; 57: ii43-ii50.
Calabrese L H, Rose-John S. IL-6 biology: implications for clinical targeting in rheumatic disease. Nat Rev Rheumatol 2014; 10:720-7.
Hunter C A, Jones S A. IL-6 as a keystone cytokine in health and disease. Nat Immunol 2015; 16:448-57.
Choy E H S, Calabrese L H. Neuroendocrine and neurophysiological effects of interleukin 6 in rheumatoid arthritis. Rheumatology (Oxford) 2018; 57:1885-95.
Solomon D H, Goodson N J, Katz J N et al. Patterns of cardiovascular risk in rheumatoid arthritis. Ann Rheum Dis 2006; 65:1608-12.
Choy E, Ganeshalingam K, Semb A G, Szekanecz Z, Nurmohamed M. Cardiovascular risk in rheumatoid arthritis: recent advances in the understanding of the pivotal role of inflammation, risk predictors and the impact of treatment. Rheumatology (Oxford) 2014; 53:2143-54.
Gabay C, McInnes I B, Kavanaugh A et al. Comparison of lipid and lipid-associated cardiovascular risk marker changes after treatment with tocilizumab or adalimumab in patients with rheumatoid arthritis. Ann Rheum Dis 2016; 75:1806-12.
Genovese M C, Fleischmann R, Kivitz A J et al. Sarilumab plus methotrexate in patients with active rheumatoid arthritis and inadequate response to methotrexate: results of a phase III study. Arthritis Rheumatol 2015; 67:1424-37.
Fleischmann R, van Adelsberg J, Lin Y et al. Sarilumab and nonbiologic disease-modifying antirheumatic drugs in patients with active rheumatoid arthritis and inadequate response or intolerance to tumor necrosis factor inhibitors. Arthritis Rheumatol 2017; 69:277-90.
Burmester G R, Lin Y, Patel R et al. Efficacy and safety of sarilumab monotherapy versus adalimumab monotherapy for the treatment of patients with active rheumatoid arthritis (MONARCH): a randomised, double-blind, parallel-group phase III trial. Ann Rheum Dis 2017; 76:840-7.
AbbVie Inc. US prescribing information: HUMIRA (adalimumab) injection, solution for subcutaneous use. 2018: https://www.accessdata.fda.gov/drugsatfda_docs/label/2008/125057s0110lbl.pdf (8 Feb. 2019, date last accessed).
Strand V, Gossec L, Proudfoot C W J et al. Patient-reported outcomes from a randomized phase III trial of sarilumab monotherapy versus adalimumab monotherapy in patients with rheumatoid arthritis. Arthritis Res Ther 2018; 20:129.
Gabay C, Msihid J, Zilberstein M et al. Identification of sarilumab pharmacodynamic and predictive markers in patients with inadequate response to TNF inhibition: a biomarker substudy of the phase 3 TARGET study. RMD Open 2018; 4: e000607.
Boyapati A, Msihid J, Fiore S, van Adelsberg J, Graham N M, Hamilton J D. Sarilumab plus methotrexate suppresses circulating biomarkers of bone resorption and synovial damage in patients with rheumatoid arthritis and inadequate response to methotrexate: a biomarker study of MOBILITY. Arthritis Res Ther 2016; 18:225.
Song S-NJ, Iwahashi M, Tomosugi N et al. Comparative evaluation of the effects of treatment with tocilizumab and TNF-α inhibitors on serum hepcidin, anemia response and disease activity in rheumatoid arthritis patients. Arthritis Res Ther 2013; 15: R141.
Gavrilă BI, Ciofu C, Stoica V. Biomarkers in rheumatoid arthritis, what is new? J Med Life 2016; 9:144-8.
Sechidis K, Papangelou K, Metcalfe P D, Svensson D, Weatherall J, Brown G. Distinguishing prognostic and predictive biomarkers: an information theoretic approach. Bioinformatics 2018; 34:3365-76.
Kringelbach™, Glintborg B, Hogdall E V, Johansen J S, Hetland M L. Identification of new biomarkers to promote personalised treatment of patients with inflammatory rheumatic disease: protocol for an open cohort study. BMJ Open 2018; 8: e019325.
Fleischmann R, Connolly S E, Maldonado M A, Schiff M. Brief report: estimating disease activity using multi-biomarker disease activity scores in rheumatoid arthritis patients treated with abatacept or adalimumab. Arthritis Rheumatol. 2016; 68:2083-9.
Curtis J R, Wright G C, Strand V, Davis C S, Hitraya E, Sasso E H. Reanalysis of the multi-biomarker disease activity score for assessing disease activity in the abatacept versus adalimumab comparison in biologic-naive rheumatoid arthritis subjects with background methotrexate study: comment on the article by Fleischmann et al. Arthritis Rheumatol. 2017; 69:863-5.
Dennis G, Jr., Holweg C T, Kummerfeld S K et al. Synovial phenotypes in rheumatoid arthritis correlate with response to biologic therapeutics. Arthritis Res Ther 2014; 16: R90.
Burmester G R, Hagino O, Dong Q et al. Unique changes in hemoglobin with sarilumab versus adalimumab are independent of better disease control in patients with rheumatoid arthritis (RA). Arthritis Rheumatol 2018; 70 (Suppl. 10): Abstract 1528.

1-87. (canceled)

88. A method of treating rheumatoid arthritis comprising:
administering, to a subject with rheumatoid arthritis whose serum sample is determined to have a pre-dose (baseline) serum concentration of hepcidin greater than 17 ng/mL, an anti-interleukin 6 receptor (anti-IL-6R) antibody,
wherein the anti-IL-6R antibody specifically binds human interleukin-6 receptor (IL-6R) and comprises three heavy chain complementarity determining regions (HCDRs) of a heavy chain variable region comprising the amino acid sequence of SEQ ID NO:1, and three light chain CDRs (LCDRs) of a light chain variable region comprising the amino acid sequence of SEQ ID NO:2.

89. The method of claim 88, wherein the HCDRs comprise HCDR1, HCDR2, and HCDR3, and the LCDRs comprise LCDR1, LCDR2, and LCDR3, wherein the HCDR1 comprises the amino acid sequence of SEQ ID NO: 3, the HCDR2 comprises the amino acid sequence of SEQ ID NO: 4, the HCDR3 comprises the amino acid sequence of SEQ ID NO: 5, the LCDR1 comprises the amino acid sequence of SEQ ID NO: 6, the LCDR2 comprises the amino acid sequence of SEQ ID NO: 7, and the LCDR3 comprises the amino acid sequence of SEQ ID NO: 8.

90. The method of claim 88, wherein the anti-IL-6R antibody comprises a heavy chain variable region comprising the amino acid sequence of SEQ ID NO: 1 and a light chain variable region comprising the amino acid sequence of SEQ ID NO: 2.

91. The method of claim 88, wherein the anti-IL-6R antibody comprises a heavy chain comprising the amino acid sequence of SEQ ID NO: 9 and a light chain comprising the amino acid sequence of SEQ ID NO: 10.

92. The method of claim 88, wherein the anti-IL-6R antibody is sarilumab.

93. The method of claim 88, wherein the subject achieves, after at least 24 weeks of treatment with the anti-IL-6R antibody:
(a) an American College of Rheumatology 20% (ACR20); and/or
(b) an improvement in at least one Patient-Reported Outcome selected from: Patient Global Visual Analogue Scale (VAS) score, Pain VAS score, morning stiffness VAS score, and Rheumatoid Arthritis Impact of Disease (RAID) score.

94. The method of claim 88, wherein the subject (i) has moderate to severe rheumatoid arthritis, and/or (ii) has had an inadequate response or is intolerant to one or more disease-modifying antirheumatic drugs (DMARDs) other than the anti-IL-6R antibody.

95. The method of claim 94, wherein the subject has had an inadequate response or is intolerant to one or more disease-modifying antirheumatic drugs (DMARDs) comprising methotrexate.

96. The method of claim 88, wherein the pre-dose (baseline) serum concentration of hepcidin is greater than 28.9 ng/ml.

97. The method of claim 88, wherein the pre-dose (baseline) serum concentration of hepcidin is greater than 43.9 ng/ml.

98. The method of claim 88, wherein the anti-IL-6R antibody is administered to the subject subcutaneously at a dose of about 150 mg once every two weeks.

99. The method of claim 88, wherein the anti-IL-6R antibody is administered to the subject subcutaneously at a dose of about 200 mg once every two weeks.

100. A method of selecting a subject with rheumatoid arthritis (RA) who has an increased propensity for effective treatment with an anti-interleukin 6 receptor (anti-IL-6R) antibody comprising:
measuring a pre-dose (baseline) serum concentration of hepcidin in a serum sample from a subject with RA,
wherein if the serum sample has a pre-dose (baseline) serum concentration of hepcidin greater than 17 ng/mL, the subject is selected as having an increased propensity for effective treatment with an anti-IL-6R antibody,
wherein the anti-IL-6R antibody specifically binds IL-6R and comprises three heavy chain complementarity determining regions (HCDRs) of a heavy chain variable region comprising the amino acid sequence of SEQ ID NO:1, and three light chain CDRs (LCDRs) of a light chain variable region comprising the amino acid sequence of SEQ ID NO: 2.

101. The method of claim 100, wherein the HCDRs comprise HCDR1, HCDR2, and HCDR3, and the LCDRs comprise LCDR1, LCDR2, and LCDR3, wherein the HCDR1 comprises the amino acid sequence of SEQ ID NO: 3, the HCDR2 comprises the amino acid sequence of SEQ ID NO: 4, the HCDR3 comprises the amino acid sequence of SEQ ID NO: 5, the LCDR1 comprises the amino acid sequence of SEQ ID NO: 6, the LCDR2 comprises the amino acid sequence of SEQ ID NO: 7, and the LCDR3 comprises the amino acid sequence of SEQ ID NO: 8.

102. The method of claim 100, wherein the anti-IL-6R antibody comprises a heavy chain variable region comprising the amino acid sequence of SEQ ID NO: 1 and a light chain variable region comprising the amino acid sequence of SEQ ID NO: 2.

103. The method of claim 100, wherein the anti-IL-6R antibody comprises a heavy chain comprising the amino acid sequence of SEQ ID NO: 9 and a light chain comprising the amino acid sequence of SEQ ID NO: 10.

104. The method of claim 100, wherein the anti-IL-6R antibody is sarilumab.

105. The method of claim 100, wherein the pre-dose (baseline) serum concentration of hepcidin is greater than 43.9 ng/ml.

106. The method of claim 100, wherein the subject who is selected as having an increased propensity for effective treatment of RA with the anti-IL-6R antibody has an increased propensity of achieving, after at least 24 weeks of treatment with the anti-IL-6R antibody:
(a) an American College of Rheumatology 20% (ACR20); and/or
(b) an improvement in at least one Patient-Reported Outcome selected from: Patient Global Visual Analogue Scale (VAS) score, Pain VAS score, morning stiffness VAS score, and Rheumatoid Arthritis Impact of Disease Score (RAID) score.

107. The method of claim 100, further comprising administering to the selected subject the anti-IL-6R antibody subcutaneously a dose of about 150 mg to about 200 mg once every two weeks.


SEQUENCE LISTING (25 sequences)

SEQ ID NO: 1 - protein, 116 aa, synthetic construct
  REGION 1..116: note = heavy chain variable region
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 2 - protein, 107 aa, synthetic construct
  REGION 1..107: note = light chain variable region
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 3 - protein, 8 aa, synthetic construct
  REGION 1..8: note = Heavy chain complementarity determining region 1
RFTFDDYA

SEQ ID NO: 4 - protein, 8 aa, synthetic construct
  REGION 1..8: note = Heavy chain complementarity determining region 2
ISWNSGRI

SEQ ID NO: 5 - protein, 9 aa, synthetic construct
  REGION 1..9: note = Heavy chain complementarity determining region 3
AKGRDSFDI

SEQ ID NO: 6 - protein, 6 aa, synthetic construct
  REGION 1..6: note = Light chain complementarity determining region 1
QGISSW

SEQ ID NO: 7 - skipped

SEQ ID NO: 8 - protein, 9 aa, synthetic construct
  REGION 1..9: note = Light chain complementarity determining region 3
QQANSFPYT

SEQ ID NO: 9 - protein, 446 aa, synthetic construct
  REGION 1..446: note = Heavy chain
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = Light chain
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 675 bp, synthetic construct
  misc_feature 1..675: note = CRP transcript variant 2 cDNA sequence (GenBank reference: NP_001315986.1)
  source 1..675: mol_type = other DNA
atggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctga

SEQ ID NO: 12 - protein, 224 aa, synthetic construct
  REGION 1..224: note = CRP peptide sequence
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 13 - DNA, 2020 bp, synthetic construct
  misc_feature 1..2020: note = CRP genomic DNA sequence (GenBank reference: NG_013007.1)
  source 1..2020: mol_type = other DNA
gactggattcagagactcaaacaatgttattgaggcatggtctatctctcagctctactcgtgagtcaaggatggtgtattagttggttttcacactgctgtaaagaactacctgagtatgggtaatttataaacaaaagaaattttaaatgaacttacagttccacatgtttggggaggactcatgaaacttacaatcatggtggaaggtgaaggggaagcaggcattttcttcacaaggcagcaggagagagacagtgtgagtgggggactgccaagcacttttatttaaatcatcagacctagtgagaactcattatcatgagcacagcatgggcaaaactacctccacgatccaatcttctcccaccatgtccctccctcaactcatggggattacaatttgagatgacatttgggtgggaacacagaaccaaaccatatcattccacctctggctcctccaaaatatcatgttcttttcacatttcaaaaccaatcataccttcccaacagtcacccaaacttaactcatttcagcattaactcaaaagtccaagtctaaagttccatctgagaaaaggcaagtcacttctgcctattagcctagtaaaataaaaaacaagttagttacttccaagatacagtgggggtataggcattgggtaaatggtcctgtttgaaatgggagaaattggccaaaacaaaggggccacaggccccatgtaaatccaaaatctggcaggacactcatgaaatcttaaagctccaaaataatctcctttgattctttgtctcacatccagggcatgctgatgcaagcggtaggcttccatggccttgggtagctccatacttgtggctcttcagggtacagcccctgtggctgctttcacaggctggcattgaacacttgcaagcttttctaagcacaaggtgcaaactgtcagtggttctaccattctgggatctggaggacagtggccctcttctcacagatccactaggcagtgccccagtggggactctgtgtggagactccaaccccacatttccctgctgcattgccctagtagaggttttctgtgagggctccatgcctgcagaagacttctgcctgaacatccaggtgtttccatacatcttctgaaatctagacagaaactcccaaagctcaactcttgtcttctgtgcatctgcaccctcaacactacttggaagccaccaaggcttggggcttgtgccctctgaagcaatggcctgagctatatacattgcccccttttagccatggctggagccgcagcagctggcacacagggtgccatgttcctgggctgcacagagcagcggggccctgggcctggcccatgataccattttttcctcctaggcttttggacctctgatgggaggggctgccatgaagatcttctgaaatgacctgaagacattttcctcattgttttggctatcaacattcatctcctcattacttatgcaaatttctgcagccagcttgaatttttccccagaaaatgggtttttcttttctaccacatggtcaggctgcacattttccaaacttttatgctccctttcccttttaaacataagttccaatttcagatcatctctttgtgaacacatatgattgtatgttttcagaaaaagccaggtcacttcttgaatgctttggtgcttagaaatttcttaagcaccaaagcattcaagaaatcatgtctcttaagttaaaagttccacagatctctagggcatgggcaaaatgccaccattgtctttgctaaaacatagaaagagtgacctttactcccgtttccaataagttcctcatctccatctaaggacacctctgcatgaacttcattttccatatcactatcagcattttggtcaaaaccattcaacaaaactcaggaagttccaagcttttccacatcttcctgtcttctcctgagccctccaaactcttccagcctctgcccctagttggttccaa

SEQ ID NO: 14 - protein, 477 aa, synthetic construct
  REGION 1..477: note = MMP-3 peptide sequence
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 15 - DNA, 1434 bp, synthetic construct
  misc_feature 1..1434: note = MMP-3 cDNA sequence
  source 1..1434: mol_type = other DNA
atgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttga

SEQ ID NO: 16 - DNA, 1822 bp, synthetic construct
  misc_feature 1..1822: note = MMP-3 mRNA sequence (GenBank reference: NM_002422.5)
  source 1..1822: mol_type = other DNA
acaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggctatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttatttta

SEQ ID NO: 17 - protein, 109 aa, synthetic construct
  REGION 1..109: note = CXCL13 peptide sequence (GenBank reference: NP_006410.1)
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 18 - DNA, 330 bp, synthetic construct
  misc_feature 1..330: note = CXCL13 cDNA sequence (GenBank reference: NP_006410.1)
  source 1..330: mol_type = other DNA
atgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctga

SEQ ID NO: 19 - DNA, 1219 bp, synthetic construct
  misc_feature 1..1219: note = CXCL13 genomic DNA sequence (GenBank reference: NM_006419.2)
  source 1..1219: mol_type = other DNA
gagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccaggaaaaagaacttccccatacaaataagcatgagactatgtaaaaataaccttgcagaagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtggggggcggggccggggggactctggtatctaattctttaatgattcctataaatctaatgacattcaataaagttgagcaaacattttacttaaaaaaaaaaaaaaaaaa

SEQ ID NO: 20 - protein, 130 aa, synthetic construct
  REGION 1..130: note = Serum amyloid A peptide sequence (GenBank reference: AAB24060.1)
MRLFTGIVFCSLVMGVTSESWRSFFKEALQGVGDMGRAYWDIMISNHQNSNRYLYARGNYDAAQRGPGGVWAAKLISRSRVYLQGLIDYYLFGNSSTVLEDSKSNEKAEEWGRSGKDPDRFRPDGLPKKY

SEQ ID NO: 21 - DNA, 393 bp, synthetic construct
  misc_feature 1..393: note = Serum amyloid A cDNA sequence (GenBank reference: AAB24060.1)
  source 1..393: mol_type = other DNA
atgaggcttttcacaggcattgttttctgctccttggtcatgggagtcaccagtgaaagctggcgttcgtttttcaaggaggctctccaaggggttggggacatgggcagagcctattgggacataatgatatccaatcaccaaaattcaaacagatatctctatgctcggggaaactatgatgctgcccaaagaggacctgggggtgtctgggctgctaaactcatcagccgttccagggtctatcttcagggattaatagactactatttatttggaaacagcagcactgtattggaggactcgaagtccaacgagaaagctgaggaatggggccggagtggcaaagaccccgaccgcttcagacctgacggcctgcctaagaaatactga

SEQ ID NO: 22 - DNA, 614 bp, synthetic construct
  misc_feature 1..614: note = Serum amyloid A mRNA sequence (GenBank reference: M81349.1)
  source 1..614: mol_type = other DNA
tatagctccacggccagaagataccagcagctctgcctttactgaaatttcagctggagaaaggtccacagcacaatgaggcttttcacaggcattgttttctgctccttggtcatgggagtcaccagtgaaagctggcgttcgtttttcaaggaggctctccaaggggttggggacatgggcagagcctattgggacataatgatatccaatcaccaaaattcaaacagatatctctatgctcggggaaactatgatgctgcccaaagaggacctgggggtgtctgggctgctaaactcatcagccgttccagggtctatcttcagggattaatagactactatttatttggaaacagcagcactgtattggaggactcgaagtccaacgagaaagctgaggaatggggccggagtggcaaagaccccgaccgcttcagacctgacggcctgcctaagaaatactgagcttcctgctcctctgctctcagggaaactgggctgtgagccacacacttctccccccagacagggacacagggtcactgagctttgtgtccccaggaactggtatagggcacctagaggtgttcaataaatgtttgtcaaattga

SEQ ID NO: 23 - protein, 532 aa, synthetic construct
  REGION 1..532: note = sICAM-1 peptide sequence (GenBank reference: NP_000192.2)
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 24 - DNA, 1598 bp, synthetic construct
  misc_feature 1..1598: note = sICAM-1 cDNA sequence (GenBank reference: NP_000192.2)
  source 1..1598: mol_type = other DNA
atggctcccagcagcccccggcccgcgctgcccgcactcctggtcctgctcggggctctgttcccaggacctggcaatgcccagacatctgtgtccccctcaaaagtcatcctgccccggggaggctccgtgctggtgactgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctga

SEQ ID NO: 25 - DNA, 2967 bp, synthetic construct
  misc_feature 1..2967: note = sICAM-1 mRNA sequence (GenBank reference: NM_000201.3)
  source 1..2967: mol_type = other DNA
gagctcctctgctactcagagttgcaacctcagcctcgctatggctcccagcagcccccggcccgcgctgcccgcactcctggtcctgctcggggctctgttcccaggacctggcaatgcccagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcaaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctgaacctatcccgggacagggcctcttcctcggccttcccatattggtggcagtggtgccacactgaacagagtggaagacatatgccatgcagctacacctaccggccctgggacgccggaggacagggcattgtcctcagtcagatacaacagcatttggggccatggtacctgcacacctaaaacactaggccacgcatctgatctgtagtcacatgactaagccaagaggaaggagcaagactcaagacatgattgatggatgttaaagtctagcctgatgagaggggaagtggtgggggagacatagccccaccatgaggacatacaactgggaaatactgaaacttgctgcctattgggtatgctgaggccccacagacttacagaagaagtggccctccatagacatgtgtagcatcaaaacacaaaggcccacacttcctgacggatgccagcttgggcactgctgtctactgaccccaacccttgatgatatgtatttattcatttgttattttaccagctatttattgagtgtcttttatgtaggctaaatgaacataggtctctggcctcacggagctcccagtcctaatcacattcaaggtcaccaggtacagttgtacaggttgtacactgcaggagagtgcctggcaaaaagatcaaatggggctgggacttctcattggccaacctgcctttccccagaaggagtgatttttctatcggcacaaaagcactatatggactggtaatggttacaggttcagagattacccagtgaggccttattcctcccttccccccaaaactgacacctttgttagccacctccccacccacatacatttctgccagtgttcacaatgacactcagcggtcatgtctggacatgagtgcccagggaatatgcccaagctatgccttgtcctcttgtcctgtttgcatttcactgggagcttgcactatgcagctccagtttcctgcagtgatcagggtcctgcaagcagtggggaagggggccaaggtattggaggactccctcccagctttggaagcctcatccgcgtgtgtgtgtgtgtgtatgtgtagacaagctctcgctctgtcacccaggctggagtgcagtggtgcaatcatggttcactgcagtcttgaccttttgggctcaagtgatcctcccacctcagcctcctgagtagctgggaccataggctcacaacaccacacctggcaaatttgattttttttttttttccagagacggggtctcgcaacattgcccagacttcctttgtgttagttaataaagctttctcaactgcc